SEQUENCE LISTING (6 sequences)

SEQ ID NO: 1 - DNA, 1776 bp, Drosophila melanogaster
cgaactttcaacactactccaagcaggccggtaatttcatatacgaattttatgcttagcaacttatttaagcccagtaaacaactagtaagccactgaaaagatcgcacaagagtacaactcccgaccagtgatacagtagacagaatcaaaagcacaaaatggccgagaccgagaaaatcgaggttcgcgacgtgactcgcatcgagcgcattggcgcccattcgcatatccgcggattgggactggacgatgtgctggaggctcgtctggtatcccagggaatggtgggccagaaggacgcgcgccgtgccgccggcgttgtggtgcagatggttcgcgagggcaagatcgccggaagatgtatcctattggccggggagcctagtaccggcaaaacggccattgctgtgggaatggcgcaggctctgggcaccgagaccccattcactagcatgtccggatcggagatatactcgctggagatgagcaagaccgaggctctgtcacaggcactgcgcaagagcattggcgttcgcatcaaggaggaaaccgagatcatcgagggcgaagtggtggagatccagatcgaacgccccgcctcgggtaccggacagaaggtgggcaaggtcaccctcaagaccaccgagatggaaaccaactacgatctgggcaacaagatcatcgagtgcttcatgaaagagaagatccaggctggcgatgtgatcaccatcgacaaggcgtccggaaaggtcaacaagctgggtcgcagcttcaccagagccagggactacgacgccactggcgctcagaccagattcgtccaatgccccgagggtgagcttcaaaaacgcaaggaggtggtgcacactgtgaccctacacgagatcgatgttatcaatagtcgcacccacgggttcttggccctgttctccggcgatactggagagatcaagcaggaggttcgcgatcagatcaacaacaaggttctcgagtggcgcgaggagggcaaagctgagataaatccgggagtactcttcatagacgaggtgcacatgctggacattgagtgcttctccttcctgaatcgcgccctggagtcggacatggctccggtggtggtgatggccaccaaccgcggcatcactcgtattaggggcactaactatcgcagtccgcacggcatacccattgatctactcgatcgcatgatcatcatacgcactgtaccgtattccgagaaggaggttaaggagatcctaaagattcgctgcgaggaggaggactgcatcatgcacccggatgccctgaccattcttacacgcatcgccacagataccagtttacgctacgccatccaactgattaccacagccaacttggtctgtcgtcgccgcaaggccaccgaagtcaataccgaggatgtgaagaaggtctactcgctcttcctggacgagaatcgctcgagcaagatcctcaaggagtaccaggatgactacatgttcagcgagatcaccgaggaggtggaaagggacccggccgctggaggcggggcaaagcgtcgcgtggagggcggcggaggagatgcccagcccatggagcactagagtctaaactgacatcgcagcaaccccccagtacattctcatgactattttatgatcaataaataagtttccttgtatctatgattaaattaaatgcctacgaatttggtcatggttttataacgattgtaattaataaaccattatcagcaaaaaaaaaaaaaaaaaa

SEQ ID NO: 2 - protein, 481 aa, Drosophila melanogaster
MAETEKIEVRDVTRIERIGAHSHIRGLGLDDVLEARLVSQGMVGQKDARRAAGVVVQMVREGKIAGRCILLAGEPSTGKTAIAVGMAQALGTETPFTSMSGSEIYSLEMSKTEALSQALRKSIGVRIKEETEIIEGEVVEIQIERPASGTGQKVGKVTLKTTEMETNYDLGNKIIECFMKEKIQAGDVITIDKASGKVNKLGRSFTRARDYDATGAQTRFVQCPEGELQKRKEVVHTVTLHEIDVINSRTHGFLALFSGDTGEIKQEVRDQINNKVLEWREEGKAEINPGVLFIDEVHMLDIECFSFLNRALESDMAPVVVMATNRGITRIRGTNYRSPHGIPIDLLDRMIIIRTVPYSEKEVKEILKIRCEEEDCIMHPDALTILTRIATDTSLRYAIQLITTANLVCRRRKATEVNTEDVKKVYSLFLDENRSSKILKEYQDDYMFSEITEEVERDPAAGGGAKRRVEGGGGDAQPMEH

SEQ ID NO: 3 - DNA, 1066 bp, Drosophila Melanogaster
ttcggcacgaggcacgaacatcgaactttagctccgctccggacacgcagtagctaaataacaaactcattactagtatattactgccgccgatttgcaaacgcgtaccgatcccgataccaggccaatcgcactccccagttcgaatcaagcaggaaaataccggataataattggcaaagatgcagatcaaagaattccgtgtgactttgccattgactgtggaagagtatcaagttgcacaattattctcggtggccgaggcgtcaaaggagaatacgggtggcggcgagggcatcgaggtgttaaaaaacgaacccttcgaagattttcccctgctgggtggcaaatacaattccggtcaatatacatataagatctaccatctgcaatcaaaagttccagcctacataagactattggcacccaagggctcattggagatccacgaggaggcatggaatgcctatccctattgtcgaacgattatcacgaaccccaagtttatgaaagatgctttcaaaataatcatcgacactctgcacgtcggagatgcgggcgattcagaaaatgtgcacgagctgacgccggataagctgaaagtgcgagagatagtgcacatcgacattgccaacgatccggtgctgcccgcggactacaagcccgatgaggatccaaccacctaccagtcaaagaagacgggccgcggtcccctggtgggatccgactggaaaaagcatgttaatcctgtcatgacctgctacaagctggtcacgtgcgagttcaaatggttcggcctgcaaacaagagtagagaatttcatacagaaatcggagcgtcgcctctttacaaacttccatcgccaagttttctgttcaaccgatcgctggtacggtctaacaatggaggacattcgcgccatcgaggaccagacgaaggaggagctggacaaggcgcggcaggtgggcgaggtgcggggtatgcgcgcggatgccgattaagtctagtagaaaattgtaaacaaaatatgtgtatgtaaaaatcaggcaaaaaaaaaaaaaaaaaa

SEQ ID NO: 4 - protein, 272 aa, Drosophila melanogaster
MQIKEFRVTLPLTVEEYQVAQLFSVAEASKENTGGGEGIEVLKNEPFEDFPLLGGKYNSGQYTYKIYHLQSKVPAYIRLLAPKGSLEIHEEAWNAYPYCRTIITNPKFMKDAFKIIIDTLHVGDAGDSENVHELTPDKLKVREIVHIDIANDPVLPADYKPDEDPTTYQSKKTGRGPLVGSDWKKHVNPVMTCYKLVTCEFKWFGLQTRVENFIQKSERRLFTNFHRQVFCSTDRWYGLTMEDIRAIEDQTKEELDKARQVGEVRGMRADAD

SEQ ID NO: 5 - DNA, 2060 bp, Drosophila melanogaster
ttcggcacgaggccgcaatgagtttgtacgattaaaagtttatgtctattcgcgtttttcgaagctttcccgattcccgtagctgtcccactgtacagcttgccacacgatgcgtccgttctccggcagcgattgccttaagcccgtcaccgagggcatcaaccgggcgttcggcgccaaggagccctggcaggtggccaccatcacggccaccacggtgctgggaggcgtctggctctggactgtgatctgccaggatgaaaatctttacattcgtggcaagcgtcagttctttaagtttgccaagaagattccagccgtgcgtcgtcaggtggagactgaattggccaaggccaaaaacgacttcgagacggaaatcaaaaagagcaacgcccaccttacctactcggaaactctgcccgagaagggactcagcaaggaggagatcctccgactggtggatgagcacctgaagactggtcactacaactggcgtgatggtcgtgtatctggcgcggtctacggctacaagcctgatctggtggagctcgtcactgaagtgtacggcaaggcctcctacaccaatcccttgcacgcagatcttttcccgggagtttgcaaaatggaggcggaggtagtgcgcatggcatgcaacctgttccatggaaactcagccagctgtggaaccatgaccaccggcggcaccgaatccattgtaatggccatgaaggcgtacagggatttcgctagagagtacaagggaatcaccaggccaaacatcgtggtgcctaagacggtccacgcggccttcgacaagggcggtcagtactttaatatccacgtgcgatccgtggatgtagatccggagacctacgaagtggacattaagaagttcaaacgtgccattaacaggaacacgattctgctggttgggtctgctccgaacttcccctatggaaccatcgatgacatcgaagctatcgccgctttgggcgttaagtacgacattcccgtgcacgtggacgcctgcctgggcagctttgtggtggccttggtccgcaacgccggctataagctgcgtcccttcgactttgaggtcaagggagtgaccagtatctccgctgatacccacaagtatggtttcgcgcccaagggatcatcggtgatcctttactcggacaagaagtacaaggaccatcagttcactgtgactactgactggcctggcggcgtgtatggttctcccacagtcaacggttcccgtgccggaggtattatcgccgcctgctgggctaccatgatgagctttggctatgatggttatctggaagccactaagcgcattgtggatacggcgcgctatatcgagaggggcgttcgcgacatcgatggcatctttatctttggcaagccagctacttcagtgattgccctgggttccaatgtgtttgacattttccggctatcggattcgctgtgcaaactgggctggaacctgaatgcgctgcagtttccatctggtatccacctgtgcgtgacggacatgcacacacagcccggagtcgcggataaattcattgccgatgtgcgcagctgtacggcggagatcatgaaggatcccggccagcccgtcgttggaaagatggctctctacggcatggcacagagcatacccgaccgttcggtgatcggagaagtgactcgcctattcctgcactccatgtactacactcccagccagaaatagacacctggagcaatccccgttctcttcgcccaccccacggagctaatgcatttcctgtgctgtatttaaaccaccaaaacaccccgtcgttaaaccttcctcaagcaatttatattaggatgcaattagtgctgtaatcgagggtacaaaacgtcgttctacgcgaaaatctatctacctatgttcatcccatttgtcaacattcgtcgctctaagagccatgttattaaagtgtttttctgtgtaacttgctagtgaaataataatataatattaatcaatttttgtgtactataaaaaaaaaaaaaaaaaa

SEQ ID NO: 6 - protein, 545 aa, Drosophila melanogaster
MRPFSGSDCLKPVTEGINRAFGAKEPWQVATITATTVLGGVWLWTVICQDENLYIRGKRQFFKFAKKIPAVRRQVETELAKAKNDFETEIKKSNAHLTYSETLPEKGLSKEEILRLVDEHLKTGHYNWRDGRVSGAVYGYKPDLVELVTEVYGKASYTNPLHADLFPGVCKMEAEVVRMACNLFHGNSASCGTMTTGGTESIVMAMKAYRDFAREYKGITRPNIVVPKTVHAAFDKGGQYFNIHVRSVDVDPETYEVDIKKFKRAINRNTILLVGSAPNFPYGTIDDIEAIAALGVKYDIPVHVDACLGSFVVALVRNAGYKLRPFDFEVKGVTSISADTHKYGFAPKGSSVILYSDKKYKDHQFTVTTDWPGGVYGSPTVNGSRAGGIIAACWATMMSFGYDGYLEATKRIVDTARYIERGVRDIDGIFIFGKPATSVIALGSNVFDIFRLSDSLCKLGWNLNALQFPSGIHLCVTDMHTQPGVADKFIADVRSCTAEIMKDPGQPVVGKMALYGMAQSIPDRSVIGEVTRLFLHSMYYTPSQK

(19) United States
(12) Patent Application Publication
(10) Pub. No.: US 2003/0217376 A1
(43) Pub. Date: Nov. 20, 2003

(54) Insecticide targets and methods of use

(76) Inventors: Allen James Ebens JR., San Carlos, CA (US); Kevin Patrick Keegan, San Diego, CA (US); Thomas J Stout, San Francisco, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/149,165

(22) PCT Filed: Dec. 7, 2000
(86) PCT No.: PCT/US00/33320

(30) Foreign Application Priority Data
Dec. 8, 1999 (US) 60169610
Dec. 28, 1999 (US) 60173228
Dec. 28, 1999 (US) 60173349

(51) Int. Cl. A01K 67/033; A01N 61/00; C07H 21/04; C12N 9/88; C12P 21/02; C12N 5/06

(52) U.S. Cl. 800/8; 435/232; 435/69.1; 435/320.1; 435/348; 514/1; 536/23.2

(57) ABSTRACT

Nucleic acids isolated from Drosophila melanogaster that are lethal when knocked out in Drosophila, and proteins encoded thereby, are described. The nucleic acids and proteins can be used to genetically modify metazoan invertebrate organisms, such as insects and worms, or cultured cells, resulting in expression or mis-expression of the encoded proteins. The genetically modified organisms or cells can be used in screening assays to identify candidate compounds which are potential pesticidal agents or therapeutics that interact with subject proteins. They can also be used in methods for studying activity of subject proteins, and identifying other genes that modulate the function of, or interact with, the subject genes.

### BACKGROUND OF THE INVENTION

Helicases are crucial to the utilization of DNA by cell metabolism. Double stranded DNA must be unwound in order to participate in such nuclear dynamics as replication, transcription and repair. This unwinding is controlled in a specific manner by a number of DNA helicases (more than 15 have been identified in yeast, bacteria and mammalian cells).

In bacteria, RuvB-like helicases are involved in complexes at Holliday junctions which include RuvA, RuvB and RuvC. RuvBs are dodecameric assemblies of two hexameric rings with ATPase activity when bound to DNA with Magnesium and ATP. TIP49b appears to be the mammalian homolog of the bacterial RuvB proteins. The RuvA-RuvB complex in the presence of ATP renatures cruciform structure in supercoiled DNA with palindromic sequence, indicating that it may promote strand exchange reactions in homologous recombination. RuvB mediates the Holliday junction migration by localized denaturation and re-annealing.

RuvB catalyzes homologous recombination and double-strand break repair. When double-strand breaks occur in DNA (by X-ray radiation or nuclease activity), the DNA ends are processed by RecBCD and introduced into homologous sequences in a heterologous duplex by RecA (Kowalczykowski et al., Microbiol. Rev. (1994) 58:401-465.). This mechanism forms a homologous recombination-directed intermediate having a four-way junction, namely the Holliday structure. In the late-stage of homologous recombination, RuvB binds to the Holliday structure, and a branch point migrates dependent on the DNA helicase activity of RuvB. Then RuvC, a Holliday structure-specific endonuclease, resolves the junction.

TIP49a and TIP49b are both mammalian homologs of bacterial RuvB, and are found in the same ˜700 kDa complex in the cell, suggesting strong evolutionary conservation of these genes. TIP49a and TIP49b share similar enzymatic properties; however, the polarity of TIP49b's helicase activity (5′ to 3′; same as RuvB) is reversed relative to TIP49a. Both TIP49a and TIP49b have been shown to be independently essential for cell growth, suggesting that their activities are not complementary. In E. coli, RuvA, RuvB and RuvC are all found sequentially on the chromosome; this does not appear to be true in eukaryotic cells.

Phospholipid transfer proteins are found in organisms from yeast to man and catalyze the transfer of phospholipids between membranes. Phophatidylinositol transfer proteins (PITPs), possess dual capability, transporting both phosphatidylinositol and phosphatidylcholine. PITP also plays essential roles in the phospholipase C- (PLC) mediated inositol lipid signaling of mammalian cells and in the formation of vesicles (Thomas et al., Cell, (1993) 74:919-928), and is necessary for regulated exocytosis (Helkamp, Subcell. Biochem. (1990) 16:129-174; Bankaitis, et al., Nature (1990) 347:561-562). The protein sequences of PITPs are highly conserved among species. Mammalian species have multiple isoforms. Alpha- and beta-isoforms of PITP share less sequence identity within a given species than each isoform shares across species, suggesting that each isoform have distinct and conserved roles. The beta isoform is capable of transferring sphingomyelin in addition to phohatidylinositol (PI) and phosphatidylcholine (PC). The alpha isoform neither binds nor transports sphingomyelin; the same is true of yeast Sec14 and the fruitfly Drosophila melanogaster (hereinafter Drosophila) protein rdgB (Westerman et al. J. Biol. Chem., (1995) 270:14263-14266).

The ability to bind and transfer PI/PC between membrane compartments defines this family of proteins. A related protein, rdgB, from Drosophila shares significant sequence homology in an N-terminal 281 amino acid domain; however, it is an integral membrane protein (1,054 amino acids) and therefore cannot carry out the transfer of lipids between membranes. Expression of that protein without the membrane anchor enables it to translocate lipids amongst membranes. The rdgb protein plays a role in the retinal degradation cascade involved in signal transduction from the retina (Vihtelic et al., J. Cell Biol.(1993) 122:1013-1022). In yeast, Sec14 has been identified as a protein with homologous function (transport of PI/PC amongst membranes), but shows no significant sequence conservation with the mammalian PITPs.

At the level of the intact organism, disruption of the expression level of PITP alpha isoform (hereinafter PITP-α) leads to neurodegeneration The “vibrator” mouse has a neurodegenerative disorder manifested by tremors that develop into an ultimately fatal, ascending motor paralysis. It has been determined that the mutant “vibrator” gene (vb) results in decreased expression of PITP-α and is the primary cause of neurodegeneration in these animals (Hamilton, Neuron, (1997) 18:711-722). Homozygous mutant mice die from apnea at post-natal day 30-160. Histological analyses indicate that the vb defect elicits a highly restricted degeneration that is limited to neurons of the spinal cord, brain stem and dorsal root ganglia. Thus, specific neuronal cells are particularly sensitive to PITP-α deficiency. How PITP-α prevents neurodegeneration remains unknown.

Deletion mutants of PITP-α have been made which impact upon the functional properties of the protein. It has been shown that the extreme C-terminus is crucial to a structural recognition event in the PLC cascade, and that lipid binding is in some manner affected by the loss of residues between 251-261 either directly or through some loss of structural integrity imperative to the lipid binding site (Prosser et al., Biochem. J., (1997) 324:19-23).

Sphingolipids and their metabolic derivatives elicit a wide variety of eukaryotic cellular responses. Although the stimuli and biological end points differ in each cell type, the role of sphingolipid by-products as second messengers in specific, growth regulatory signal transduction pathways appears to be a universal theme among eukaryotic cells (Hannun, J. Biol. Chem. (1994) 269:3125-3128). Sphingosine and sphingosine 1-phosphate (S-1-P) are both catabolites of sphingolipid breakdown, which have been shown to modulate DNA synthesis and cellular proliferation in mammalian cells (Olivera and Spiegel Nature (1993) 365:557-559). Evidence suggests that S-1-P is largely responsible for these effects. In addition, S-1-P has recently been shown to inhibit the growth, motility, and invasiveness of tumor cells (Sadahira et al., Proc. Natl. Acad. Sci. U.S.A. (1992) 89:9686-9690; Spiegel et al., Breast Cancer Res. Treat. (1994) 31:337-348). Free sphingosine and S-1-P are maintained at very low levels in mammalian cells (Merrll et al., Anal. Biochem. (1988) 171:373-381). This is consistent with the notion that potent second messengers are tightly regulated in the absence of a particular stimulus. The mechanism(s) by which the intracellular levels of sphingosine and S-1-P are regulated have not been established. Such control may occur at the synthetic stage, via regulation of the activities of ceramidases and sphingosine kinase (Buehrer and Bell, Adv. Lipid Res. (1993) 26:59-67). Alternatively, control may occur at the catabolic stage, through regulation of the activity of sphingosine phosphate lyase (SPL) (Veldhoven and Mannaerts, Adv. Lipid Res. (1993) 26:69-98). Sphingolipids exist in yeast where they provide vital, yet unknown functions (Wells, and Lester, J. Biol. Chem. (1983) 258, 10200-10203). S-1-P has also been shown to be associated with the enhanced expression of the Bax protein, which is involvedin apoptosis (Hung and Chuang, Biochem. Biophys. Res. Comm. (1996) 229:11-15). S-1-P blocks cell death induced by ceramide and tumor necrosis factor-alpha (Cuvillier et al., Nature (1996) 81:800-803).

Pesticide development has traditionally focused on the chemical and physical properties of the pesticide itself, a relatively time-consuming and expensive process. As a consequence, efforts have been concentrated on the modification of pre-existing, well-validated compounds, rather than on the development of new pesticides.

There is a need in the art for new pesticidal compounds that are safer, more selective, and more efficient than currently available pesticides. The present invention addresses this need by providing novel pesticide targets from invertebrates such as the fruit fly Drosophila melanogaster, and by providing methods of identifying compounds that bind to and modulate the activity of such targets.

### SUMMARY OF THE INVENTION

It is an object of the invention to provide insect nucleic acids and proteins that are targets for pesticides. The insect nucleic acid molecules provided herein are useful for producing insect proteins encoded thereby. The insect proteins are useful in assays to identify compounds that modulate a biological activity of the proteins, which assays identify compounds that may have utility as pesticides.

It is an object of the present invention to provide invertebrate homologs of a Helicase, hereinafter referred to as dmHelicase, that can be used in genetic screening methods to characterize pathways that dmHelicase may be involved in as well as other interacting genetic pathways. It is also an object of the invention to provide methods for screening compounds that interact with dmHelicase such as those that may have utility as therapeutics or pesticides.

It is a further object of the present invention to provide invertebrate homologs of a PITP, hereinafter referred to as dmPITP, that can be used in genetic screening methods to characterize pathways that dmPITP may be involved in as well as other interacting genetic pathways. It is also an object of the invention to provide methods for screening compounds that interact with dmPITP such as those that may have utility as therapeutics or pesticides.

It is a further object of the present invention to provide invertebrate homologs of a SPL gene, hereinafter referred to as dmSPL1, that can be used in genetic screening methods to characterize pathways that dmSPL1 may be involved in as well as other interacting genetic pathways. It is also an object of the invention to provide methods for screening compounds that interact with dmSPL1 such as those that may have utility as therapeutics or pesticides.

These and other objects are provided by the present invention, which concerns the identification and characterization of novel pesticidal targets in Drosophila melanogaster that are lethal when knocked out in Drosophila. Isolated nucleic acid molecules are provided that comprise nucleic acid sequences encoding target proteins as well as novel fragments and derivatives thereof. Methods of using the isolated nucleic acid molecules and fragments of the invention as biopesticides are described, such as use of RNA interference methods that block a biological activity of the target protein. Vectors and host cells comprising the subject nucleic acid molecules are also described, as well as metazoan invertebrate organisms (e.g. insects, coelomates and pseudocoelomates) that are genetically modified to express or mis-express a subject protein.

An important utility of the novel target nucleic acids and proteins is that they can be used in screening assays to identify candidate compounds which are potential pesticidal agents or therapeutics that interact with a target protein. Such assays typically comprise contacting a subject protein or fragment with one or more candidate molecules, and detecting any interaction between the candidate compound and the subject protein. The assays may comprise adding the candidate molecules to cultures of cells genetically engineered to express subject proteins, or alternatively, administering the candidate compound to a metazoan invertebrate organism genetically engineered to express a subject protein.

The genetically engineered metazoan invertebrate animals of the invention can also be used in methods for studying a biological activity of a subject protein. These methods typically involve detecting the phenotype caused by the expression or mis-expression of the subject protein. The methods may additionally comprise observing a second animal that has the same genetic modification as the first animal and, additionally has a mutation in a gene of interest. Any difference between the phenotypes of the two animals identifies the gene of interest as capable of modifying the function of the gene encoding the subject protein.

### DETAILED DESCRIPTION OF THE INVENTION

The use of invertebrate model organism genetics and related technologies can greatly facilitate the elucidation of biological pathways (Scangos, Nat. Biotechnol. (1997) 15:1220-1221; Margolis and Duyk, supra). Of particular use is the insect model organism, Drosophila melanogaster (hereinafter referred to generally as “Drosophila”). An extensive search for Helicase nucleic acids and their encoded proteins in Drosophila was conducted in an attempt to identify new and useful tools for probing the function and regulation of the Helicase genes, and for use as targets in pesticide and drug discovery.

Novel insect nucleic acid molecules, and proteins encoded thereby, are provided herein. Novel nucleic acids and their encoded proteins are identified herein. The Drosophila target nucleic acids and proteins presented here were identified via mutation to lethality by P-element transposon insertion, discussed in more detail below. The P-element lethality, along with the DNA processing functions, identifies the subject Drosophila proteins as previously unrecognized insecticidal drug targets for antagonist drugs. The newly identified nucleic acids can be used for the generation of mutant phenotypes in animal models or in living cells that can be used to study regulation of proteins encoded by the subject nucleic acid molecules, and the use of subject proteins as pesticide or drug targets. Due to the ability to rapidly carry out large-scale, systematic genetic screens, the use of invertebrate model organisms such as Drosophila has great utility for analyzing the expression and mis-expression of a subject protein. Thus, the invention provides a superior approach for identifying other components involved in the synthesis, activity, and regulation of the subject proteins. Systematic genetic analysis of the subject proteins using invertebrate model organisms can lead to the identification and validation of pesticide targets directed to components of biochemical pathways involving the subject proteins. Model organisms or cultured cells that have been genetically engineered to express the subject proteins can be used to screen candidate compounds for their ability to modulate subject protein expression or activity, and thus are useful in the identification of new drug targets, therapeutic agents, diagnostics and prognostics useful in the treatment of disorders associated with DNA processing. Additionally, these invertebrate model organisms can be used for the identification and screening of pesticide targets directed to components of a pathway involving a subject protein.

The details of the conditions used for the identification and/or isolation of novel subject nucleic acid and protein are described in the Examples section below. Various non-limiting embodiments of the invention, applications and uses of these novel gene and protein are discussed in the following sections. The entire contents of all references, including patent applications, cited herein are incorporated by reference in their entireties for all purposes. Additionally, the citation of a reference in the preceding background section is not an admission of prior art against the claims appended hereto.

For the purposes of the present application, singular forms “a”, “and”, and “the” include plural referents unless the context clearly indicates otherwise. Thus, for example, reference to “an invertebrate receptor” includes large numbers of receptors, reference to “an agent” includes large numbers of agents and mixtures thereof, reference to “the method” includes one or more methods or steps of the type described herein.

Definitions

As used herein the term “isolated” is meant to describe a polynucleotide, a polypeptide, an antibody, or a host cell that is in an environment different from that in which the polynucleotide, the polypeptide, the antibody, or the host cell naturally occurs. As used herein, the term “substantially purified” refers to a compound (e.g., either a polynucleotide or a polypeptide or an antibody) that is removed from its natural environment and is at least 60% free, preferably 75% free, and most preferably 90% free from other components with which it is naturally associated.

A “host cell”, as used herein, denotes microorganisms or eukaryotic cells or cell lines cultured as unicellular entities which can be, or have been, used as recipients for recombinant vectors or other transfer polynucleotides, and include the progeny of the original cell which has been transfected. It is understood that the progeny of a single cell may not necessarily be completely identical in morphology or in genomic or total DNA complement as the original parent, due to natural, accidental, or deliberate mutation.

By “transformation” is meant a permanent or transient genetic change induced in a cell following incorporation of new DNA (i.e., DNA exogenous to the cell). Genetic change can be accomplished either by incorporation of the new DNA into the genome of the host cell, or by transient or stable maintenance of the new DNA as an episomal element. Where the cell is a eukaryotic cell, a permanent genetic change is generally achieved by introduction of the DNA into the genome of the cell.

Isolated Nucleic Acids of the Invention

The present invention provides isolated nucleic acid molecules that comprise nucleotide sequences encoding insect proteins that are potential pesticide targets. The isolated nucleic acid molecules have a variety of uses, e.g., as hybridization probes, e.g., to identify nucleic acid molecules that share nucleotide sequence identity; in expression vectors to produce the polypeptides encoded by the nucleic acid molecules; and to modify a host cell or animal for use in assays described hereinbelow.

Thus, the term “isolated nucleic acid sequence”, as used herein, includes the reverse complement, RNA equivalent, DNA or RNA single- or double-stranded sequences, and DNA/RNA hybrids of the sequence being described, unless otherwise indicated.

The terms “polynucleotide” and “nucleic acid”, used interchangeably herein, refer to a polymeric forms of nucleotides of any length, either ribonucleotides or deoxynucleotides. Thus, this tem includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidites and thus can be an oligodeoxynucleoside phosphoramidate or a mixed phosphoramidate-phosphodiester oligomer. Peyrottes et al. (1996) Nucl. Acids Res. 24:1841-1848; Chaturvedi et al. (1996) Nucl. Acids Res. 24:2318-2323. A polynucleotide may comprise modified nucleotides, such as methylated nucleotides and nucleotide analogs, uracyl, other sugars, and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support.

For hybridization probes, it may be desirable to use nucleic acid analogs, in order to improve the stability and and binding affinity. A number of modifications have been described that alter the chemistry of the phosphodiester backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire phosphodiester backbone with a peptide linkage.

Sugar modifications are also used to enhance stability and affinity. The a-anomer of deoxyribose may be used, where the base is inverted with respect to the natural b-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

Derivative nucleic acid sequences of the subject nucleic acid molecules include sequences that hybridize to the nucleic acid sequence of any one of SEQ ID NOS:1, 3, or 5 under stringency conditions such that the hybridizing derivative nucleic acid is related to the subject nucleic acid by a certain degree of sequence identity. A nucleic acid molecule is “hybridizable” to another nucleic acid molecule, such as a cDNA, genomic DNA, or RNA, when a single stranded form of the nucleic acid molecule can anneal to the other nucleic acid molecule. Stringency of hybridization refers to conditions under which nucleic acids are hybridizable. The degree of stringency can be controlled by temperature, ionic strength, pH, and the presence of denaturing agents such as formamide during hybridization and washing. As used herein, the term “stringent hybridization conditions” are those normally used by one of skill in the art to establish at least a 90% sequence identity between complementary pieces of DNA or DNA and RNA. “Moderately stringent hybridization conditions” are used to find derivatives having at least 70% sequence identity. Finally, “low-stringency hybridization conditions” are used to isolate derivative nucleic acid molecules that share at least about 50% sequence identity with the subject nucleic acid sequence.

The ultimate hybridization stringency reflects both the actual hybridization conditions as well as the washing conditions following the hybridization, and it is well known in the art how to vary the conditions to obtain the desired result. Conditions routinely used are set out in readily available procedure texts (e.g., Current Protocols in Molecular Biology, Vol. 1, Chap. 2.10, John Wiley & Sons, Publishers (1994); Sambrook et al., Molecular Cloning, Cold Spring Harbor (1989)). A preferred derivative nucleic acid is capable of hybridizing to SEQ ID NO:1 under stringent hybridization conditions that comprise: prehybridization of filters containing nucleic acid for 8 hours to overnight at 65° C. in a solution comprising 6× single strength citrate (SSC) (1×SSC is 0.15 M NaCl, 0.015 M Na citrate; pH 7.0), 5× Denhardt's solution, 0.05% sodium pyrophosphate and 100 μg/ml herring sperm DNA; hybridization for 18-20 hours at 65° C. in a solution containing 6×SSC, 1× Denhardt's solution, 100 μg/ml yeast tRNA and 0.05% sodium pyrophosphate; and washing of filters at 65° C. for 1 h in a solution containing 0.2×SSC and 0.1% SDS (sodium dodecyl sulfate).

Fragments of the subject nucleic acid molecules can be used for a variety of purposes. Interfering RNA (RNAi) fragments, particularly double-stranded (ds) RNAi, can be used to generate loss-of-function phenotypes, or to formulate biopesticides (discussed further below). Fragments of the subject nucleic acid molecules are also useful as nucleic acid hybridization probes and replication/amplification primers. Certain “antisense” fragments, i.e. that are reverse complements of portions of the coding sequence of the subject nucleic acid sequences have utility in inhibiting the function of proteins encoded by the subject nucleic acid molecules. The fragments are of length sufficient to specifically hybridize with the corresponding subject nucleic acid molecule. The fragments generally consist of or comprise at least 12, preferably at least 24, more preferably at least 36, and more preferably at least 96 contiguous nucleotides of a subject nucleic acid molecule. When the fragments are flanked by other nucleic acid sequences, the total length of the combined nucleic acid sequence is less than 15 kb, preferably less than 10 kb or less than 5 kb, more preferably less than 2 kb, and in some cases, preferably less than 500 bases.

The subject nucleic acid sequences and fragments thereof may be joined to other components such as labels, peptides, agents that facilitate transport across cell membranes, hybridization-triggered cleavage agents or intercalating agents. The subject nucleic acid sequences and fragments thereof may also be joined to other nucleic acid sequences (i.e. they may comprise part of larger sequences) and are of synthetic/non-natural sequences and/or are isolated and/or are purified, i.e. unaccompanied by at least some of the material with which it is associated in its natural state. Preferably, the isolated nucleic acids constitute at least about 0.5%, and more preferably at least about 5% by weight of the total nucleic acid present in a given fraction, and are preferably recombinant, meaning that they comprise a non-natural sequence or a natural sequence joined to nucleotide(s) other than that which it is joined to on a natural chromosome.

Derivative nucleic acid sequences that have at least about 70% sequence identity with one of SEQ ID NOS:1, 3, or 5 are capable of hybridizing to one of SEQ ID NOS:1, 3, or 5 under moderately stringent conditions that comprise: pretreatment of filters containing nucleic acid for 6 hours at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA; hybridization for 18-20 h at 40° C. in a solution containing 35% formamide, 5×SSC, 50 mM Tris-HCl (pH 7.5), 5 mM EDTA, 0.02% PVP, 0.02% Ficoll, 0.2% BSA, 100 μg/ml salmon sperm DNA, and 10% (wt/vol) dextran sulfate; followed by washing twice for 1 hour at 55° C. in a solution containing 2×SSC and 0.1% SDS.

Other preferred derivative nucleic acid sequences are capable of hybridizing to one of SEQ ID NOS:1, 3, or 5 under low stringency conditions that comprise: incubation for 8 hours to overnight at 37° C. in a solution comprising 20% formamide, 5×SSC, 50 mM sodium phosphate (pH 7.6), 5× Denhardt's solution, 10% dextran sulfate, and 20 μg/ml denatured sheared salmon sperm DNA; hybridization in the same buffer for 18 to 20 hours; and washing of filters in 1×SSC at about 37° C. for 1 hour.

As used herein, “percent (%) nucleic acid sequence identity” with respect to a subject sequence, or a specified portion of a subject sequence, is defined as the percentage of nucleotides in the candidate derivative nucleic acid sequence identical with the nucleotides in the subject sequence (or specified portion thereof), after aligning the sequences and introducing gaps, if necessary to achieve the maximum percent sequence identity, as generated by the program WU-BLAST-2.0a19 (Altschul et al., J. Mol. Biol. (1997) 215:403-410; http://blast.wustl.edu/blast/README.html; hereinafter referred to generally as “BLAST”) with all the search parameters set to default values. The HSP S and HSP S2 parameters are dynamic values and are established by the program itself depending upon the composition of the particular sequence and composition of the particular database against which the sequence of interest is being searched. A percent (%) nucleic acid sequence identity value is determined by the number of matching identical nucleotides divided by the sequence length for which the percent identity is being reported.

Another type of derivative of the subject nucleic acid sequences includes corresponding humanized sequences. A humanized nucleic acid sequence is one in which one or more codons has been substituted with a codon that is more commonly used in human genes. Preferably, a sufficient number of codons have been substituted such that a higher level expression is achieved in mammalian cells than what would otherwise be achieved without the substitutions. Tables are available in the art that show, for each amino acid, the calculated codon frequency in humans genes for 1000 codons (Wada et al., Nucleic Acids Research (1990) 18(Suppl.):2367-2411). Similarly, other nucleic acid derivatives can be generated with codon usage optimized for expression in other organisms, such as yeasts, bacteria, and plants, where it is desired to engineer the expression of receptor proteins by using specific codons chosen according to the preferred codons used in highly expressed genes in each organism. A detailed discussion of the humanization of nucleic acid sequences is provided in U.S. Pat. No. 5,874,304 to Zolotukhin et al.

A derivative invertebrate target nucleic acid sequence, or fragment thereof, may comprise 100% sequence identity with any one of SEQ ID NOS:1, 3, or 5 but be a derivative thereof in the sense that it has one or more modifications at the base or sugar moiety, or phosphate backbone. Examples of modifications are well known in the art (Bailey, Ullmann's Encyclopedia of Industrial Chemistry (1998), 6th ed. Wiley and Sons). Such derivatives may be used to provide modified stability or any other desired property.

Exemplary target nucleic acid molecules of the invention are described in detail below.

dmHelicase Nucleic Acids

In some embodiments, the invention provides nucleic acid sequences of Helicases, and more particularly Helicase nucleic acid sequences of Drosophila, and methods of using these sequences. As described in the Examples below, a nucleic acid sequence (SEQ ID NO:1) was isolated from Drosophila that encodes a Helicase homolog, hereinafter referred to as dmHelicase. In addition to the fragments and derivatives of SEQ ID NO: 1 as described in detail below, the invention includes the reverse complements thereof Also, the subject nucleic acid sequences, derivatives and fragments thereof may be RNA molecules comprising the nucleotide sequence of SEQ ID NO: 1 (or derivative or fragment thereof) wherein the base U (uracil) is substituted for the base T (thymine). The DNA and RNA sequences of the invention can be single- or double-stranded. Thus, the term “isolated nucleic acid sequence”, as used herein, includes the reverse complement, RNA equivalent, DNA or RNA single- or double-stranded sequences, and DNA/RNA hybrids of the sequence being described, unless otherwise indicated.

In some embodiments, a dmHelicase nucleic acid molecule comprises at least about 20, at least about 50, at least about 75, at least about 100, at least about 150, at least about 200, at least about 300, at least about 400, at least about 500, at least about 600, at least about 700, at least about 800, at least about 900, at least about 1000, at least about 1100, at least about 1200, at least about 1300, at least about 1400, at least about 1500, at least about 1600, at least about 1700, or at least about 1750 contiguous nucleotides of the sequence set forth in SEQ ID NO:1, up to the entire sequence set forth in SEQ ID NO:1.

In other embodiments, a dmHelicase nucleic acid molecule of the invention comprises a nucleotide sequence that encodes a polypeptide comprising at least about 6, at least about 10, at least about 20, at least about 50, at least about 75, at least about 100, at least about 150, at least about 200, at least about 250, at least about 300, at least about 350, at least about 400, at least about 450, or at least about 475 contiguous amino acids of the sequence set forth in SEQ ID NO:2, up to the entire amino acid sequence as set forth in SEQ ID NO:2.

A preferred fragment of SEQ ID NO:1 comprises nucleotides 380-401, which encode an ATP/GTP binding site motif A.

Derivative dmHelicase nucleic acid sequences usually have at least 80% sequence identity, preferably at least 85% sequence identity, more preferably at least 90% sequence identity, still more preferably at least 95% sequence identity, and most preferably at least 98% sequence identity with SEQ ID NO:1.

In one preferred embodiment, the derivative nucleic acid encodes a polypeptide comprising a dmHelicase amino acid sequence of SEQ ID NO:2, or a fragment or derivative thereof as described further below under the subheading “dmHelicase proteins”.

More specific embodiments of preferred dmHelicase protein fragments and derivatives are discussed further below in connection with specific dmHelicase proteins.

dmPITP Nucleic Acid Molecules

In some embodiments, the invention provides nucleic acid sequences of PITPs, and more particularly PITP nucleic acid sequences of Drosophila, and methods of using these sequences. As described in the Examples below, a nucleic acid sequence (SEQ ID NO:3) was isolated from Drosophila that encodes a PITP homolog, hereinafter referred to as dmPITP. In addition to the fragments and derivatives of SEQ ID NO:3 as described in detail below, the invention includes the reverse complements thereof.

In some embodiments, a dmPITP nucleic acid molecule of the invention comprises at least about 20, at least about 50, at least about 75, at least about 100, at least about 150, at least about 200, at least about 300, at least about 400, at least about 500, at least about 600, at least about 700, at least about 800, at least about 900, at least about 1000, or at least about 1050 contiguous nucleotides of the sequence set forth in SEQ ID NO:3, up to the entire sequence set forth in SEQ ID NO:3.

In other embodiments, a dmPITP nucleic acid molecule of the invention comprises a nucleotide sequence that encodes a polypeptide comprising at least about 6, at least about 10, at least about 20, at least about 50, at least about 75, at least about 100, at least about 150, at least about 200, at least about 250, or at least about 270 contiguous amino acids of the sequence set forth in SEQ ID NO:4, up to the entire amino acid sequence as set forth in SEQ ID NO:4.

Derivative dmPITP nucleic acid sequences usually have at least 70% sequence identity, preferably at least 80% sequence identity, more preferably at least 85% sequence identity, still more preferably at least 90% sequence identity, and most preferably at least 95% sequence identity with SEQ ID NO:1, or domain-encoding regions thereof.

In one preferred embodiment, the derivative nucleic acid encodes a polypeptide comprising a dmPITP amino acid sequence of SEQ ID NO:2, or a fragment or derivative thereof as described further below under the subheading “dmPITP proteins”.

More specific embodiments of preferred dmPITP protein fragments and derivatives are discussed further below in connection with specific dmPITP proteins.

dmSPL Nucleic Acid Molecules

In some embodiments, the invention provides nucleic acid sequences of SPLs, and more particularly SPL nucleic acid sequences of Drosophila, and methods of using these sequences. As described in the Examples below, a nucleic acid sequence (SEQ ID NO:5) was isolated from Drosophila that encodes a SPL homolog, hereinafter referred to dmSPL1. In addition to the fragments and derivatives of SEQ ID NO:5 as described in detail below, the invention includes the reverse complements thereof.

In some embodiments, a dmSPL nucleic acid molecule comprises at least about 20, at least about 50, at least about 75, at least about 100, at least about 150, at least about 200, at least about 300, at least about 400, at least about 500, at least about 600, at least about 700, at least about 800, at least about 900, at least about 1000, at least about 1100, at least about 1200, at least about 1300, at least about 1400, at least about 1500, at least about 1600, at least about 1700, at least about 1800, at least about 1900, at least about 2000, or at least about 2050 contiguous nucleotides of the sequence set forth in SEQ ID NO:5, up to the entire sequence set forth in SEQ ID NO:5.

In other embodiments, a dmSPL nucleic acid molecule of the invention comprises a nucleotide sequence that encodes a polypeptide comprising at least about 6, at least about 10, at least about 20, at least about 50, at least about 75, at least about 100, at least about 150, at least about 200, at least about 250, at least about 300, at least about 350, at least about 400, at least about 450, at least about 500, or at least about 545 contiguous amino acids of the sequence set forth in SEQ ID NO:6.

Additional preferred fragments of SEQ ID NO:5 encode extracellular or intracellular domains, which are located at approximately nucleotides 110-1008, and 1058-1744.

Derivative dmSPL1 nucleic acid sequences usually have at least 70% sequence identity, preferably at least 80% sequence identity, more preferably at least 85% sequence identity, still more preferably at least 90% sequence identity, and most preferably at least 95% sequence identity with SEQ ID NO:5, or domain-encoding regions thereof.

More specific embodiments of preferred dmSPL 1 protein fragments and derivatives are discussed further below in connection with specific dmSPL1 proteins.

Isolation, Production, and Expression of Subject Nucleic Acids

Nucleic acid encoding the amino acid sequence of any of SEQ ID NOS:2, 4, or 6, or fragment or derivative thereof, may be obtained from an appropriate cDNA library prepared from any eukaryotic species that encodes a subject protein such as vertebrates, preferably mammalian (e.g. primate, porcine, bovine, feline, equine, and canine species, etc.) and invertebrates, such as arthropods, particularly insects species (preferably Drosophila), acarids, crustacea, molluscs, nematodes, and other worms. An expression library can be constructed using known methods. For example, mRNA can be isolated to make cDNA which is ligated into a suitable expression vector for expression in a host cell into which it is introduced. Various screening assays can then be used to select for the gene or gene product (e.g. oligonucleotides of at least about 20 to 80 bases designed to identify the gene of interest, or labeled antibodies that specifically bind to the gene product). The gene and/or gene product can then be recovered from the host cell using known techniques.

Polymerase chain reaction (PCR) can also be used to isolate nucleic acids of the subject proteins, where oligonucleotide primers representing fragmentary sequences of interest amplify RNA or DNA sequences from a source such as a genomic or cDNA library (as described by Sambrook et al., supra). Additionally, degenerate primers for amplifying homologs from any species of interest may be used. Once a PCR product of appropriate size and sequence is obtained, it may be cloned and sequenced by standard techniques, and utilized as a probe to isolate a complete cDNA or genomic clone.

Fragmentary sequences of the subject nucleic acids and derivatives may be synthesized by known methods. For example, oligonucleotides may be synthesized using an automated DNA synthesizer available from commercial suppliers (e.g. Biosearch, Novato, Calif.; Perkin-Elmer Applied Biosystems, Foster City, Calif.). Antisense RNA sequences can be produced intracellularly by transcription from an exogenous sequence, e.g. from vectors that contain antisense nucleic acid sequences. Newly generated sequences may be identified and isolated using standard methods.

A subject isolated nucleic acid sequence can be inserted into any appropriate cloning vector, for example bacteriophages such as lambda derivatives, or plasmids such as pBR322, pUC plasmid derivatives and the Bluescript vector (Stratagene, San Diego, Calif.). Recombinant molecules can be introduced into host cells via transformation, transfection, infection, electroporation, etc., or into a transgenic animal such as a fly. The transformed cells can be cultured to generate large quantities of a subject nucleic acid. Suitable methods for isolating and producing the subject nucleic acid sequences are well-known in the art (Sambrook et al., supra; DNA Cloning: A Practical Approach, Vol. 1, 2, 3, 4, (1995) Glover, ed., MRL Press, Ltd., Oxford, U.K).

The nucleotide sequence encoding a subject protein or fragment or derivative thereof, can be inserted into any appropriate expression vector for the transcription and translation of the inserted protein-coding sequence. Alternatively, the necessary transcriptional and translational signals can be supplied by the native subject gene and/or its flanking regions. A variety of host-vector systems may be utilized to express the protein-coding sequence such as mammalian cell systems infected with virus (e.g. vaccinia virus, adenovirus, etc.); insect cell systems infected with virus (e.g. baculovirus); microorganisms such as yeast containing yeast vectors, or bacteria transformed with bacteriophage, DNA, plasmid DNA, or cosmid DNA. Expression of a subject protein may be controlled by a suitable promoter/enhancer element. In addition, a host cell strain may be selected which modulates the expression of the inserted sequences, or modifies and processes the gene product in the specific fashion desired.

To detect expression of the subject gene product, the expression vector can comprise a promoter operably linked to a subject gene nucleic acid, one or more origins of replication, and, one or more selectable markers (e.g. thymidine kinase activity, resistance to antibiotics, etc.). Alternatively, recombinant expression vectors can be identified by assaying for the expression of a subject gene product based on the physical or functional properties of a subject protein in in vitro assay systems (e.g. immunoassays).

The subject proteins, fragments, or derivatives may be optionally expressed as a fusion, or chimeric protein product (i.e. it is joined via a peptide bond to a heterologous protein sequence of a different protein). A chimeric product can be made by ligating the appropriate nucleic acid sequences encoding the desired amino acid sequences to each other in the proper coding frame using standard methods and expressing the chimeric product. A chimeric product may also be made by protein synthetic techniques, e.g. by use of a peptide synthesizer.

Once a recombinant that expresses a subject gene sequence is identified, the gene product can be isolated and purified using standard methods (e.g. ion exchange, affinity, and gel exclusion chromatography; centrifugation; differential solubility; electrophoresis). The amino acid sequence of the protein can be deduced from the nucleotide sequence of the chimeric gene contained in the recombinant and can thus be synthesized by standard chemical methods (Hunkapiller et al., Nature (1984) 310:105-111). Alternatively, native subject proteins can be purified from natural sources, by standard methods (e.g. immunoaffinity purification).

Target Proteins of the Invention

Purified target proteins of the invention comprise or consist of an amino acid sequence of any of SEQ ID NOS:2, 4, or 6, or fragments or derivatives thereof. Compositions comprising any of these proteins may consist essentially of a subject protein, fragments, or derivatives, or may comprise additional components (e.g. pharmaceutically acceptable carriers or excipients, culture media, carriers used in pesticide formulations, etc.).

Derivatives of the subject proteins typically share a certain degree of sequence identity or sequence similarity with any of SEQ ID NOS:2, 4, or 6, or a fragment thereof. As used herein, “percent (%) amino acid sequence identity” with respect to a subject sequence, or a specified portion of a subject sequence, is defined as the percentage of amino acids in the candidate derivative amino acid sequence identical with the amino acid in the subject sequence (or specified portion thereof), after aligning the sequences and introducing gaps, if necessary to achieve the maximum percent sequence identity, as generated by BLAST (Altschul et al., supra) using the same parameters discussed above for derivative nucleic acid sequences. A % amino acid sequence identity value is determined by the number of matching identical amino acids divided by the sequence length for which the percent identity is being reported. “Percent (%) amino acid sequence similarity” is determined by doing the same calculation as for determining % amino acid sequence identity, but including conservative amino acid substitutions in addition to identical amino acids in the computation. A conservative amino acid substitution is one in which an amino acid is substituted for another amino acid having similar properties such that the folding or activity of the protein is not significantly affected. Aromatic amino acids that can be substituted for each other are phenylalanine, tryptophan, and tyrosine; interchangeable hydrophobic amino acids are leucine, isoleucine and valine; interchangeable polar amino acids are glutamine and asparagine; interchangeable basic amino acids arginine, lysine and histidine; interchangeable acidic amino acids aspartic acid and glutamic acid; and interchangeable small amino acids alanine, serine, threonine, methionine, and glycine.

The fragment or derivative of a subject protein is preferably “functionally active” meaning that the subject protein derivative or fragment exhibits one or more functional activities associated with a full-length, wild-type subject protein comprising the amino acid sequence of any of SEQ ID NOS:2, 4, or 6. As one example, a fragment or derivative may have antigenicity such that it can be used in immunoassays, for immunization, for inhibition of activity of a subject protein, etc, as discussed further below regarding generation of antibodies to subject proteins. Preferably, a functionally active fragment or derivative of a subject protein is one that displays one or more biological activities associated with a subject protein, such as enzymatic activity. For purposes herein, functionally active fragments also include those fragments that exhibit one or more structural features of a subject protein, such as an ATP/GTP binding domain. The functional activity of the subject proteins, derivatives and fragments can be assayed by various methods known to one skilled in the art (Current Protocols in Protein Science (1998) Coligan et al., eds., John Wiley & Sons, Inc., Somerset, N.J.). In a preferred method, which is described in detail below, a model organism, such as Drosophila, is used in genetic studies to assess the phenotypic effect of a fragment or derivative (i.e. a mutant subject protein).

Derivatives of the subject proteins can be produced by various methods known in the art. The manipulations that result in their production can occur at the gene or protein level. For example, a cloned subject gene sequence can be cleaved at appropriate sites with restriction endonuclease(s) (Wells et al., Philos. Trans. R. Soc. London SerA (1986) 317:415), followed by further enzymatic modification if desired, isolated, and ligated in vitro, and expressed to produce the desired derivative. Alternatively, a subject gene can be mutated in vitro or in vivo, to create and/or destroy translation, initiation, and/or termination sequences, or to create variations in coding regions and/or to form new restriction endonuclease sites or destroy preexisting ones, to facilitate further in vitro modification. A variety of mutagenesis techniques are known in the art such as chemical mutagenesis, in vitro site-directed mutagenesis (Carter et al., Nucl. Acids Res. (1986) 13:4331), use of TAB® linkers (available from Pharmacia and Upjohn, Kalamazoo, Mich.), etc.

At the protein level, manipulations include post translational modification, e.g. glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known technique (e.g. specific chemical cleavage by cyanogen bromide, trypsin, chymotrypsin, papain, V8 protease, NaBH₄, acetylation, formylation, oxidation, reduction, metabolic synthesis in the presence of tunicamycin, etc.). Derivative proteins can also be chemically synthesized by use of a peptide synthesizer, for example to introduce nonclassical amino acids or chemical amino acid analogs as substitutions or additions into a subject protein sequence.

Chimeric or fusion proteins can be made comprising a subject protein or fragment thereof (preferably comprising one or more structural or functional domains of a subject protein) joined at its amino- or carboxy-terminus via a peptide bond to an amino acid sequence of a different protein. Chimeric proteins can be produced by any known method, including: recombinant expression of a nucleic acid encoding the protein (comprising a coding sequence encoding a subject protein joined in-frame to a coding sequence for a different protein); ligating the appropriate nucleic acid sequences encoding the desired amino acid sequences to each other in the proper coding frame, and expressing the chimeric product; and protein synthetic techniques, e.g. by use of a peptide synthesizer.

dmHelicase Protein

In some embodiments, the invention provides dmHelicase proteins, or fragments or derivatives thereof.

In other embodiments, a dmHelicase protein or fragment of the invention comprises an amino acid sequence of at least about 24, at least about 26, at least about 29, at least about 34, at least about 50, at least about 75, at least about 80, at least about 100, at least about 150, at least about 200, at least about 250, at least about 300, at least about 350, at least about 400, at least about 450, or at least about 475 contiguous amino acids of the sequence set forth in SEQ ID NO:2, up to the entire amino acid sequence as set forth in SEQ ID NO:2.

In one preferred embodiment, a subject protein derivative shares at least 80% sequence identity or similarity, preferably at least 85%, more preferably at least 90%, and most preferably at least 95% sequence identity or similarity with a contiguous stretch of at least 25 amino acids, preferably at least 50 amino acids, more preferably at least 100 amino acids, and in some cases, the entire length of SEQ ID NO:2.

In another embodiment, a subject protein derivative may consist of or comprise a sequence that shares 100% similarity with any contiguous stretch of at least 49 amino acids, preferably at least 51 amino acids, more preferably at least 54 amino acids, and most preferably at least 59 amino acids of SEQ ID NO:2. In a preferred embodiment, the dmHelicase protein or derivative thereof comprises amino acid residues 73-80, which is a putative ATP/GTP-binding site motif. Another preferred derivative of dmHelicase protein consists of or comprises a sequence of at least 26 amino acids that share 100% similarity with an equivalent number of contiguous amino acids of residues of SEQ ID NO:2.

Preferred fragments of dmHelicase proteins consist or comprise at least 24, preferably at least 26, more preferably at least 29, and most preferably at least 34 contiguous amino acids of residues 187-236 of SEQ ID NO:2.

dmPITP Proteins

In some embodiments, the invention provides dmPITP proteins, or fragments or derivatives thereof.

In other embodiments, a dmPTIP protein of fragment of the invention comprises an amino acid sequence of at least about 14, at least about 16, at least about 19, at least about 24, at least about 50, at least about 75, at least about 100, at least about 150, at least about 200, at least about 250, or at least about 270 contiguous amino acids of the sequence set forth in SEQ ID NO:4, up to the entire amino acid sequence as set forth in SEQ ID NO:4.

In one preferred embodiment, a dmPITP protein derivative shares at least 80% sequence identity or similarity, preferably at least 85%, more preferably at least 90%, and most preferably at least 95% sequence identity or similarity with a contiguous stretch of at least 25 amino acids, preferably at least 50 amino acids, more preferably at least 100 amino acids, and in some cases, the entire length of SEQ ID NO:4.

In another embodiment, the dmPITP protein derivative may consist of or comprise a sequence that shares 100% similarity with any contiguous stretch of at least 27 amino acids, preferably at least 29 amino acids, more preferably at least 32 amino acids, and most preferably at least 37 ammo acids of SEQ ID NO:4.

dmSPL Proteins

In some embodiments, the invention provides dmSPL1 proteins, or fragments or derivatives thereof.

In some embodiments, a dmSPL protein or fragment of the invention comprises an amino acid sequence of at least about 15, at least about 17, at least about 20, at least about 25, at least about 50, at least about 75, at least about 100, at least about 150, at least about 200, at least about 250, at least about 300, at least about 350, at least about 400, at least about 450, at least about 500, or at least about 545 contiguous amino acids of the sequence set forth in SEQ ID NO:6.

In one preferred embodiment, a dmSPL1 protein derivative shares at least 80% sequence identity or similarity, preferably at least 85%, more preferably at least 90%, and most preferably at least 95% sequence identity or similarity with a contiguous stretch of at least 25 amino acids, preferably at least 50 amino acids, more preferably at least 100 amino acids, and in some cases, the entire length of SEQ ID NO:6.

In another embodiment, the dmSPL1 protein derivative may consist of or comprise a sequence that shares 100% similarity with any contiguous stretch of at least 36 amino acids, preferably at least 38 amino acids, more preferably at least 41 amino acids, and most preferably at least 46 amino acids of SEQ ID NO:6. Preferred derivatives of dmSPL1 consist of or comprise an amino acid sequence that has at least 80%, preferably at least 85%, more preferably at least 90%, and most preferably at least 95% sequence identity or sequence similarity with any of amino acid residues 1-299 and 317-545, which are the likely extracellular or intracellular domains.

Gene Regulatory Elements of the Subject Nucleic Acid Molecules

The invention further provides gene regulatory DNA elements, such as enhancers or promoters that control transcription of the subject nucleic acid molecules. Such regulatory elements can be used to identify tissues, cells, genes and factors that specifically control production of a subject protein. Analyzing components that are specific to a particular subject protein function can lead to an understanding of how to manipulate these regulatory processes, especially for pesticide and therapeutic applications, as well as an understanding of how to diagnose dysfunction in these processes.

Gene fusions with the subject regulatory elements can be made. For compact genes that have relatively few and small intervening sequences, such as those described herein for Drosophila, it is typically the case that the regulatory elements that control spatial and temporal expression patterns are found in the DNA immediately upstream of the coding region, extending to the nearest neighboring gene. Regulatory regions can be used to construct gene fusions where the regulatory DNAs are operably fused to a coding region for a reporter protein whose expression is easily detected, and these constructs are introduced as transgenes into the animal of choice. An entire regulatory DNA region can be used, or the regulatory region can be divided into smaller segments to identify sub-elements that might be specific for controlling expression a given cell type or stage of development. Reporter proteins that can be used for construction of these gene fusions include E. coli beta-galactosidase and green fluorescent protein (GFP). These can be detected readily in situ, and thus are useful for histological studies and can be used to sort cells that express a subject protein (O'Kane and Gehring PNAS (1987) 84(24):9123-9127; Chalfie et al., Science (1994) 263:802-805; and Cumberledge and Krasnow (1994) Methods in Cell Biology 44:143-159). Recombinase proteins, such as FLP or cre, can be used in controlling gene expression through site-specific recombination (Golic and Lindquist (1989) Cell 59(3):499-509; White et al., Science (1996) 271:805-807). Toxic proteins such as the reaper and hid cell death proteins, are useful to specifically ablate cells that normally express a subject protein in order to assess the physiological function of the cells (Kingston, In Current Protocols in Molecular Biology (1998) Ausubel et al., John Wiley & Sons, Inc. sections 12.0.3-12.10) or any other protein where it is desired to examine the function this particular protein specifically in cells that synthesize a subject protein.

Alternatively, a binary reporter system can be used, similar to that described further below, where a subject regulatory element is operably fused to the coding region of an exogenous transcriptional activator protein, such as the GAL4 or tTA activators described below, to create a subject regulatory element “driver gene”. For the other half of the binary system the exogenous activator controls a separate “target gene” containing a coding region of a reporter protein operably fused to a cognate regulatory element for the exogenous activator protein, such as UAS_{G} or a tTA-response element, respectively. An advantage of a binary system is that a single driver gene construct can be used to activate transcription from preconstructed target genes encoding different reporter proteins, each with its own uses as delineated above.

Subject regulatory element-reporter gene fusions are also useful for tests of genetic interactions, where the objective is to identify those genes that have a specific role in controlling the expression of subject genes, or promoting the growth and differentiation of the tissues that expresses a subject protein. Subject gene regulatory DNA elements are also useful in protein-DNA binding assays to identify gene regulatory proteins that control the expression of subject genes. The gene regulatory proteins can be detected using a variety of methods that probe specific protein-DNA interactions well known to those skilled in the art (Kingston, supra) including in vivo footprinting assays based on protection of DNA sequences from chemical and enzymatic modification within living or permeabilized cells; and in vitro footprinting assays based on protection of DNA sequences from chemical or enzymatic modification using protein extracts, nitrocellulose filter-binding assays and gel electrophoresis mobility shift assays using radioactively labeled regulatory DNA elements mixed with protein extracts. Candidate gene regulatory proteins can be purified using a combination of conventional and DNA-affinity purification techniques. Molecular cloning strategies can also be used to identify proteins that specifically bind subject gene regulatory DNA elements. For example, a Drosophila cDNA library in an expression vector, can be screened for cDNAs that encode dmHelicase gene regulatory element DNA-binding activity. Similarly, the yeast “one-hybrid” system can be used (Li and Herskowitz, Science (1993) 262:1870-1874; Luo et al., Biotechniques (1996) 20(4):564-568; Vidal et al., PNAS (1996) 93(19): 10315-10320).

dmHelicase Regulatory Elements

In some embodiments, the invention provides dmHelicase regulatory elements that reside within nucleotides 1 to 161 of SEQ ID NO: 1. Preferably at least 20, more preferably at least 25, and most preferably at least 50 contiguous nucleotides within nucleotides 1 to 161 of SEQ ID NO: 1 are used.

dmPITP Regulatory Elements

In some embodiments, the invention provides dmPITP gene regulatory elements that reside within nucleotides 1 to 182 of SEQ ID NO:3. Preferably at least 20, more preferably at least 25, and most preferably at least 50 contiguous nucleotides within nucleotides 1 to 182 of SEQ ID NO:3 are used.

dmSPL Regulatory Elements

In some embodiments, the invention provides dmSPL1 gene regulatory elements, that reside within nucleotides 1 to 109 of SEQ ID NO:5. Preferably at least 20, more preferably at least 25, and most preferably at least 50 contiguous nucleotides within nucleotides 1 to 109 of SEQ ID NO:5 are used.

Antibodies to Subject Proteins

The subject proteins, fragments thereof, and derivatives thereof may be used as an immunogen to generate monoclonal or polyclonal antibodies and antibody fragments or derivatives (e.g. chimeric, single chain, Fab fragments). For example, fragments of a subject protein, preferably those identified as hydrophilic, are used as immunogens for antibody production using art-known methods such as by hybridomas; production of monoclonal antibodies in germ-free animals (PCT/US90/02545); the use of human hybridomas (Cole et al., PNAS (1983) 80:2026-2030; Cole et al., in Monoclonal Antibodies and Cancer Therapy (1985) Alan R Liss, pp. 77-96), and production of humanized antibodies (Jones et al, Nature (1986)321:522-525; U.S. Pat. No. 5,530,101). In a particular embodiment, subject polypeptide fragments provide specific antigens and/or immunogens, especially when coupled to carrier proteins. For example, peptides are covalently coupled to keyhole limpet antigen (KLH) and the conjugate is emulsified in Freund's complete adjuvant. Laboratory rabbits are immunized according to conventional protocol and bled. The presence of specific antibodies is assayed by solid phase immunosorbent assays using immobilized corresponding polypeptide. Specific activity or function of the antibodies produced may be determined by convenient in vitro, cell-based, or in vivo assays: e.g. in vitro binding assays, etc. Binding affinity may be assayed by determination of equilibrium constants of antigen-antibody association (usually at least about 10⁷ M⁻¹, preferably at least about 10⁸ M⁻¹, more preferably at least about 10⁹ M⁻¹).

Identification of Molecules that Interact with a Subject Protein

A variety of methods can be used to identify or screen for molecules, such as proteins or other molecules, that interact with a subject protein, or derivatives or fragments thereof. The assays may employ purified protein, or cell lines or model organisms such as Drosophila and C. elegans, that have been genetically engineered to express a subject protein. Suitable screening methodologies are well known in the art to test for proteins and other molecules that interact with a subject gene and protein (see e.g., PCT International Publication No. WO 96/34099). The newly identified interacting molecules may provide new targets for pharmaceutical or pesticidal agents. Any of a variety of exogenous molecules, both naturally occurring and/or synthetic (e.g., libraries of small molecules or peptides, or phage display libraries), may be screened for binding capacity. In a typical binding experiment, a subject protein or fragment is mixed with candidate molecules under conditions conducive to binding, sufficient time is allowed for any binding to occur, and assays are performed to test for bound complexes. Assays to find interacting proteins can be performed by any method known in the art, for example, immunoprecipitation with an antibody that binds to the protein in a complex followed by analysis by size fractionation of the imunoprecipitated proteins (e.g. by denaturing or nondenaturing polyacrylamide gel electrophoresis), Western analysis, non-denaturing gel electrophoresis, two-hybrid systems (Fields and Song, Nature (1989) 340:245-246; U.S. Pat. No. 5,283,173; for review see Brent and Finley, Annu. Rev. Genet. (1977) 31:663-704), etc.

Immunoassays

Immunoassays can be used to identify proteins that interact with or bind to a subject protein. Various assays are available for testing the ability of a protein to bind to or compete with binding to a wild-type subject protein or for binding to an anti-subject protein antibody. Suitable assays include radioimmunoassays, ELISA (enzyme linked immunosorbent assay), immunoradiometric assays, gel diffusion precipitin reactions, immunodiffusion assays, in situ immunoassays (e.g., using colloidal gold, enzyme or radioisotope labels), western blots, precipitation reactions, agglutination assays (e.g., gel agglutination assays, hemagglutination assays), complement fixation assays, immunofluorescence assays, protein A assays, immunoelectrophoresis assays, etc.

Identification of Potential Pesticide or Drug Targets

Once new target genes or target interacting genes are identified, they can be assessed as potential pesticide or drug targets, or as potential biopesticides. Further, transgenic plants that express subject proteins can be tested for activity against insect pests (Estruch et al., Nat. Biotechnol (1997) 15(2):137-141).

The subject proteins are validated pesticide targets, since disruption of the Drosophila the subject genes results in lethality when homozygous. The mutation to lethality of these gene indicates that drugs that agonize or antagonize the gene product may be effective pesticidal agents.

As used herein, the term “pesticide” refers generally to chemicals, biological agents, and other compounds that kill, paralyze, sterilize or otherwise disable pest species in the areas of agricultural crop protection, human and animal health. Exemplary pest species include parasites and disease vectors such as mosquitoes, fleas, ticks, parasitic nematodes, chiggers, mites, etc. Pest species also include those that are eradicated for aesthetic and hygienic purposes (e.g. ants, cockroaches, clothes moths, flour beetles, etc.), home and garden applications, and protection of structures (including wood boring pests such as termites, and marine surface fouling organisms).

Pesticidal compounds can include traditional small organic molecule pesticides (typified by compound classes such as the organophosphates, pyrethroids, carbamates, and organochlorines, benzoylureas, etc.). Other pesticides include proteinaceous toxins such as the Bacillus thuringiensis Crytoxins (Gill et al., Annu Rev Entomol (1992) 37:615-636) and Photorabdus luminescens toxins (Bowden et al., Science (1998) 280:2129-2132); and nucleic acids such as subject dsRNA or antisense nucleic acids that interferes with activity of a subject nucleic acid molecule. Pesticides can be delivered by a variety of means including direct application to pests or to their food source. In addition to direct application, toxic proteins and pesticidal nucleic acids (e.g. dsRNA) can be administered using biopesticidal methods, for example, by viral infection with nucleic acid or by transgenic plants that have been engineered to produce interfering nucleic acid sequences or encode the toxic protein, which are ingested by plant-eating pests.

Putative pesticides, drugs, and molecules can be applied onto whole insects, nematodes, and other small invertebrate metazoans, and the ability of the compounds to modulate (e.g. block or enhance) activity of a subject protein can be observed. Alternatively, the effect of various compounds on a subject protein can be assayed using cells that have been engineered to express one or more subject proteins and associated proteins.

Assays of Compounds on Worms

In a typical worm assay, the compounds to be tested are dissolved in DMSO or other organic solvent, mixed with a bacterial suspension at various test concentrations, preferably OP50 strain of bacteria (Brenner, Genetics (1974) 110:421-440), and supplied as food to the worms. The population of worms to be treated can be synchronized larvae (Sulston and Hodgkin, in the nematode C. elegans (1988), supra) or adults or a mixed-stage population of animals.

Adult and larval worms are treated with different concentrations of compounds, typically ranging from 1 mg/ml to 0.001 mg/ml. Behavioral aberrations, such as a decrease in motility and growth, and morphological aberrations, sterility, and death are examined in both acutely and chronically treated adult and larval worms. For the acute assay, larval and adult worms are examined immediately after application of the compound and re-examined periodically (every 30 minutes) for 5-6 hours. Chronic or long-term assays are performed on worms and the behavior of the treated worms is examined every 8-12 hours for 4-5 days. In some circumstances, it is necessary to reapply the pesticide to the treated worms every 24 hours for maximal effect.

Assays of Compounds on Insects

Potential insecticidal compounds can be administered to insects in a variety of ways, including orally (including addition to synthetic diet, application to plants or prey to be consumed by the test organism), topically (including spraying, direct application of compound to animal, allowing animal to contact a treated surface), or by injection. Insecticides are typically very hydrophobic molecules and must commonly be dissolved in organic solvents, which are allowed to evaporate in the case of methanol or acetone, or at low concentrations can be included to facilitate uptake (ethanol, dimethyl sulfoxide).

The first step in an insect assay is usually the determination of the minimal lethal dose (MLD) on the insects after a chronic exposure to the compounds. The compounds are usually diluted in DMSO, and applied to the food surface bearing 0-48 hour old embryos and larvae. In addition to MLD, this step allows the determination of the fraction of eggs that hatch, behavior of the larvae, such as how they move/feed compared to untreated larvae, the fraction that survive to pupate, and the fraction that eclose (emergence of the adult insect from puparium). Based on these results more detailed assays with shorter exposure times may be designed, and larvae might be dissected to look for obvious morphological defects. Once the MLD is determined, more specific acute and chronic assays can be designed.

In a typical acute assay, compounds are applied to the food surface for embryos, larvae, or adults, and the animals are observed after 2 hours and after an overnight incubation. For application on embryos, defects in development and the percent that survive to adulthood are determined. For larvae, defects in behavior, locomotion, and molting may be observed. For application on adults, behavior and neurological defects are observed, and effects on fertility are noted.

For a chronic exposure assay, adults are placed on vials containing the compounds for 48 hours, then transferred to a clean container and observed for fertility, neurological defects, and death.

Assay of Compounds using Cell Cultures

Compounds that modulate (e.g. block or enhance) a subject protein's activity may also be assayed using cell culture. For example, various compounds added to cells expressing a subject protein may be screened for their ability to modulate the activity of subject genes based upon measurements of a biological activity of a subject protein. Assays for changes in a biological activity of a subject protein can be performed on cultured cells expressing endogenous normal or mutant subject protein. Such studies also can be performed on cells transfected with vectors capable of expressing the subject protein, or functional domains of one of the subject protein, in normal or mutant form. In addition, to enhance the signal measured in such assays, cells may be cotransfected with genes encoding a subject protein.

Alternatively, cells expressing a subject protein may be lysed, the subject protein purified, and tested in vitro using methods known in the art (Kanemaki M., et al., J Biol Chem, 1999 274:22437-22444).

Compounds that selectively modulate a subject protein are identified as potential pesticide and drug candidates having specificity for the subject protein.

Identification of small molecules and compounds as potential pesticides or pharmaceutical compounds from large chemical libraries requires high-throughput screening (HTS) methods (Bolger, Drug Discovery Today (1999) 4:251-253). Several of the assays mentioned herein can lend themselves to such screening methods. For example, cells or cell lines expressing wild type or mutant subject protein or its fragments, and a reporter gene can be subjected to compounds of interest, and depending on the reporter genes, interactions can be measured using a variety of methods such as color detection, fluorescence detection (e.g. GFP), autoradiography, scintillation analysis, etc.

Subject Nucleic Acids as Biopesticides

Subject nucleic acids and fragments thereof, such as antisense sequences or double-stranded RNA (dsRNA), can be used to inhibit subject nucleic acid molecule function, and thus can be used as biopesticides. Methods of using dsRNA interference are described in published PCT application WO 99/32619. The biopesticides may comprise the nucleic acid molecule itself, an expression construct capable of expressing the nucleic acid, or organisms transfected with the expression construct. The biopesticides may be applied directly to plant parts or to soil surrounding the plants (e.g. to access plant parts growing beneath ground level), or directly onto the pest.

Biopesticides comprising a subject nucleic acid may be prepared in a suitable vector for delivery to a plant or animal. For generating plants that express the subject nucleic acids, suitable vectors include Agrobacterium tumefaciens Ti plasmid-based vectors (Horsch et al., Science (1984) 233:496-89; Fraley et al, Proc. Natl. Acad. Sci. USA (1983) 80:4803), and recombinant cauliflower mosaic virus (Hohn et al., 1982, In Molecular Biology of Plant Tumors, Academic Press, New York, pp 549-560; U.S. Pat. No. 4,407,956 to Howell). Retrovirus based vectors are useful for the introduction of genes into vertebrate animals (Burns et al., Proc. Natl. Acad. Sci. USA (1993) 90:8033-37).

Transgenic insects can be generated using a transgene comprising a subject gene operably fused to an appropriate inducible promoter. For example, a tTA-responsive promoter may be used in order to direct expression of a subject protein at an appropriate time in the life cycle of the insect. In this way, one may test efficacy as an insecticide in, for example, the larval phase of the life cycle (i.e. when feeding does the greatest damage to crops). Vectors for the introduction of genes into insects include P element (Rubin and Spradling, Science (1982) 218:348-53; U.S. Pat. No. 4,670,388), “hermes” (O'Brochta et al., Genetics (1996) 142:907-914), “minos” (U.S. Pat. No. 5,348,874), “mariner” (Robertson, Insect Physiol. (1995) 41:99-105), and “sleeping beauty” (Ivics et al., Cell (1997) 91(4):501-510), “piggyBac” (Thibault et al., Insect Mol Biol (1999) 8(1):119-23), and “hobo” (Atkinson et al., Proc. Natl. Acad. Sci. U.S.A. (1993) 90:9693-9697). Recombinant virus systems for expression of toxic proteins in infected insect cells are well known and include Semliki Forest virus (DiCionmo and Bremner, J. Biol. Chem. (1998) 273:18060-66), recombinant sindbis virus (Higgs et al., Insect Mol. Biol. (1995) 4:97-103; Seabaugh et al., Virology (1998) 243:99-112), recombinant pantropic retrovirus (Matsubara et al., Proc. Natl. Acad. Sci. USA (1996) 93:6181-85; Jordan et al., Insect Mol. Biol. (1998) 7:215-22), and recombinant baculovirus (Cory and Bishop, Mol. Biotechnol. (1997) 7(3):303-13; U.S. Pat. No. 5,470,735; U.S. Pat. Nos. 5,352,451; U.S. Pat. No. 5, 770, 192; U.S. Pat. No. 5,759,809; U.S. Pat. No. 5,665,349; and U.S. Pat. No. 5,554,592).

Generation and Genetic Analysis of Animals and Cell Lines with Altered Expression of a Subject Gene

Both genetically modified animal models (i.e. in vivo models), such as C. elegans and Drosophila, and in vitro models such as genetically engineered cell lines expressing or mis-expressing subject pathway genes, are useful for the functional analysis of these proteins. Model systems that display detectable phenotypes, can be used for the identification and characterization of subject pathway genes or other genes of interest and/or phenotypes associated with the mutation or mis-expression of subject pathway protein. The term “mis-expression” as used herein encompasses mis-expression due to gene mutations. Thus, a mis-expressed subject pathway protein may be one having an amino acid sequence that differs from wild-type (i.e. it is a derivative of the normal protein). A mis-expressed subject pathway protein may also be one in which one or more amino acids have been deleted, and thus is a “fragment” of the normal protein. As used herein, “mis-expression” also includes ectopic expression (e.g. by altering the normal spatial or temporal expression), over-expression (e.g. by multiple gene copies), underexpression, non-expression (e.g by gene knockout or blocking expression that would otherwise normally occur), and further, expression in ectopic tissues. As used in the following discussion concerning in vivo and in vitro models, the term “gene of interest” refers to a subject pathway gene, or any other gene involved in regulation or modulation, or downstream effector of the subject pathway.

The in vivo and in vitro models may be genetically engineered or modified so that they 1) have deletions and/or insertions of one or more subject pathway genes, 2) harbor interfering RNA sequences derived from subject pathway genes, 3) have had one or more endogenous subject pathway genes mutated (e.g. contain deletions, insertions, rearrangements, or point mutations in subject gene or other genes in the pathway), and/or 4) contain transgenes for mis-expression of wild-type or mutant forms of such genes. Such genetically modified in vivo and in vitro models are useful for identification of genes and proteins that are involved in the synthesis, activation, control, etc. of subject pathway gene and/or gene products, and also downstream effectors of subject function, genes regulated by subject, etc. The newly identified genes could constitute possible pesticide targets (as judged by animal model phenotypes such as non-viability, block of normal development, defective feeding, defective movement, or defective reproduction). The model systems can also be used for testing potential pesticidal or pharmaceutical compounds that interact with the subject pathway, for example by administering the compound to the model system using any suitable method (e.g. direct contact, ingestion, injection, etc.) and observing any changes in phenotype, for example defective movement, lethality, etc. Various genetic engineering and expression modification methods which can be used are well-known in the art, including chemical mutagenesis, transposon mutagenesis, antisense RNAi, dsRNAi, and transgene-mediated mis-expression.

Generating Loss-of-function Mutations by Mutagenesis

Loss-of-function mutations in an invertebrate metazoan subject gene can be generated by any of several mutagenesis methods known in the art (Ashburner, In Drosophila melanogaster: A Laboratory Manual (1989), Cold Spring Harbor, N.Y., Cold Spring Harbor Laboratory Press: pp. 299-418; Fly pushing: The Theory and Practice of Drosophila melanogaster Genetics (1997) Cold Spring Harbor Press, Plainview, N.Y.; The nematode C. elegans (1988) Wood, Ed., Cold Spring Harbor Laboratory Press, Cold Spring harbor, New York). Techniques for producing mutations in a gene or genome include use of radiation (e.g., X-ray, UV, or gamma ray); chemicals (e.g., EMS, MMS, ENU, formaldehyde, etc.); and insertional mutagenesis by mobile elements including dysgenesis induced by transposon insertions, or transposon-mediated deletions, for example, male recombination, as described below. Other methods of altering expression of genes include use of transposons (e.g., P element, EP-type “overexpression trap” element, mariner element, piggyBac transposon, hermes, minos, sleeping beauty, etc.) to misexpress genes; antisense; double-stranded RNA interference; peptide and RNA aptamers; directed deletions; homologous recombination; dominant negative alleles; and intrabodies.

Transposon insertions lying adjacent to a gene of interest can be used to generate deletions of flanking genomic DNA, which if induced in the germline, are stably propagated in subsequent generations. The utility of this technique in generating deletions has been demonstrated and is well-known in the art. One version of the technique using collections of P element transposon induced recessive lethal mutations (P lethals) is particularly suitable for rapid identification of novel, essential genes in Drosophila (Cooley et al., Science (1988) 239:1121-1128; Spralding et al., PNAS (1995) 92:0824-10830). Since the sequence of the P elements are known, the genomic sequence flanking each transposon insert is determined either by plasmid rescue (Hamilton et al., PNAS (1991) 88:2731-2735) or by inverse polymerase chain reaction, using well-established techniques. (Rehm, http://www.friuitfly.org/mlthods/). The subject genes were identified from a P lethal screen. Disruption of the Drosophila subject gene results in lethality when homozygous, indicating that this protein is critical for cell function and the survival of insects. The mutation to lethality of this gene indicates that drugs which agonize or antagonize the encoded subject protein will be effective insecticidal agents and that this class of proteins are excellent targets for drug screening and discovery.

A more recent version of the transposon insertion technique in male Drosophila using P elements is known as P-mediated male recombination (Preston and Engels, Genetics (1996) 144:1611-1638).

Generating Loss-of-Function Phenotypes Using RNA-based Methods

The subject genes may be identified and/or characterized by generating loss-of-function phenotypes in animals of interest through RNA-based methods, such as antisense RNA (Schubiger and Edgar, Methods in Cell Biology (1994) 44:697-713). One form of the antisense RNA method involves the injection of embryos with an antisense RNA that is partially homologous to the gene of interest (in this case the subject gene). Another form of the antisense RNA method involves expression of an antisense RNA partially homologous to the gene of interest by operably joining a portion of the gene of interest in the antisense orientation to a powerful promoter that can drive the expression of large quantities of antisense RNA, either generally throughout the animal or in specific tissues. Antisense RNA-generated loss-of-function phenotypes have been reported previously for several Drosophila genes including cactus, pecanex, and Krüppel (LaBonne et al., Dev. Biol. (1989) 136(1):1-16; Schuh and Jackle, Genome (1989) 31(1):422-425; Geisler et al., Cell (1992) 71(4):613-621).

Loss-of-function phenotypes can also be generated by cosuppression methods (Bingham Cell (1997) 90(3):385-387; Smyth, Curr. Biol. (1997) 7(12):793-795; Que and Jorgensen, Dev. Genet. (1998) 22(1): 100-109). Cosuppression is a phenomenon of reduced gene expression produced by expression or injection of a sense strand RNA corresponding to a partial segment of the gene of interest. Cosuppression effects have been employed extensively in plants and C. elegans to generate loss-of-function phenotypes, and there is a single report of cosuppression in Drosophila, where reduced expression of the Adh gene was induced from a white-Adh transgene using cosuppression methods (Pal-Bhadra et al., Cell (1997) 90(3):479-490).

Another method for generating loss-of-function phenotypes is by double-stranded RNA interference (dsRNAi). This method is based on the interfering properties of double-stranded RNA derived from the coding regions of gene, and has proven to be of great utility in genetic studies of C. elegans (Fire et al., Nature (1998) 391:806-811), and can also be used to generate loss-of-function phenotypes in Drosophila (Kennerdell and Carthew, Cell (1998) 95:1017-1026; Misquitta and Patterson PNAS (1999) 96:1451-1456). In one example of this method, complementary sense and antisense RNAs derived from a substantial portion of a gene of interest, such as a subject gene, are synthesized in vitro. The resulting sense and antisense RNAs are annealed in an injection buffer, and the double-stranded RNA injected or otherwise introduced into animals (such as in their food or by soaking in the buffer containing the RNA). Progeny of the injected animals are then inspected for phenotypes of interest (PCT publication no. WO99/32619).

Generating Loss-of-Function Phenotypes Using Peptide and RNA Aptamers

Additional methods that can be used for generating loss-of-function phenotypes include use of peptide aptamers that act as dominant inhibitors of protein function (Kolonin and Finley, PNAS (1998) 95:14266-14271; Xu et al., PNAS (1997) 94:12473-12478; Hoogenboomet al, Immunotechnology (1998) 4:1-20), RNA aptamers (Good et al., Gene Therapy (1997) 4:45-54; Ellington et al., Biotechnol. Annu. Rev. (1995) 1:185-214; Bell et al., J. Biol. Chem. (1998) 273:14309-14314; Shi et al., Proc. Natl. Acad. Sci USA (1999) 96:10033-10038), and intrabodies (Chen et al, Hum. Gen. Ther. (1994) 5:595-601; Hassanzadeh et al., Febs Lett. (1998) 16:75-86).

Generating Loss of Function Phenotypes Using Intrabodies

Intracellularly expressed antibodies, or intrabodies, are single-chain antibody molecules designed to specifically bind and inactivate target molecules inside cells. Intrabodies have been used in cell assays and in whole organisms such as Drosophila (Chen et al., Hum. Gen. Ther. (1994) 5:595-601; Hassanzadeh et al., Febs Lett. (1998)16(1, 2):75-80 and 81-86). Inducible expression vectors can be constructed with intrabodies that react specifically with a subject protein. These vectors can be introduced into model organisms and studied in the same manner as described above for aptamers.

Transgenesis

Typically, transgenic animals are created that contain gene fusions of the coding regions of a subject gene (from either genomic DNA or cDNA) or genes engineered to encode antisense RNAs, cosuppression RNAs, interfering dsRNA, RNA aptamers, peptide aptamers, or intrabodies operably joined to a specific promoter and transcriptional enhancer whose regulation has been well characterized, preferably heterologous promoters/enhancers (i.e. promoters/enhancers that are non-native to a subject pathway genes being expressed).

Methods are well known for incorporating exogenous nucleic acid sequences into the genome of animals or cultured cells to create transgenic animals or recombinant cell lines. For invertebrate animal models, the most common methods involve the use of transposable elements. There are several suitable transposable elements that can be used to incorporate nucleic acid sequences into the genome of model organisms. Transposable elements are particularly useful for inserting sequences into a gene of interest so that the encoded protein is not properly expressed, creating a “knock-out” animal having a loss-of-function phenotype. Techniques are well-established for the use of P element in Drosophila (Rubin and Spradling, Science (1982) 218:348-53; U.S. Pat. No. 4,670,388) and Tc1 in C. elegans (Zwaal et al., Proc. Natl. Acad. Sci. U.S.A. (1993) 90:7431-7435; and Caenorhabditis elegans: Modern Biological Analysis of an Organism (1995) Epstein and Shakes, Eds.). Other Tc1-like transposable elements can be used such as minos, mariner and sleeping beauty. Additionally, transposable elements that function in a variety of species, have been identified, such as PiggyBac (Thibault et al., Insect Mol Biol (1999) 8(1):119-23), hobo, and hermes.

P elements, or marked P elements, are preferred for the isolation of loss-of-function mutations in Drosophila genes because of the precise molecular mapping of these genes, depending on the availability and proximity of preexisting P element insertions for use as a localized transposon source (Hamilton and Zinn, Methods in Cell Biology (1994) 44:81-94; and Wolfner and Goldberg, Methods in Cell Biology (1994) 44:33-80). Typically, modified P elements are used which contain one or more elements that allow detection of animals containing the P element. Most often, marker genes are used that affect the eye color of Drosophila, such as derivatives of the Drosophila white or rosy genes (Rubin and Spradling, Science (1982) 218(4570):348-353; and Klemenz et al., Nucleic Acids Res. (1987) 15(10):3947-3959). However, in principle, any gene can be used as a marker that causes a reliable and easily scored phenotypic change in transgenic animals. Various other markers include bacterial plasmid sequences having selectable markers such as ampicillin resistance (Steller and Pirrotta, EMBO. J. (1985) 4:167-171); and lacZ sequences fused to a weak general promoter to detect the presence of enhancers with a developmental expression pattern of interest (Beflen et al., Genes Dev. (1989) 3(9): 1288-1300). Other examples of marked P elements useful for mutagenesis have been reported (Nucleic Acids Research (1998) 26:85-88; and http://flybase.bio.indiana.edu).

Preferred methods of transposon mutagenesis in Drosophila employ the “local hopping” method described by Tower et al. (Genetics (1993) 133:347-359) or generation of localized deletions from Drosophila lines carrying P insertions in the gene of interest using known methods (Kaiser, Bioassays (1990) 12(6);297-301; Harnessing the power of Drosophila genetics, In Drosophila melanogaster: Practical Uses in Cell and Molecular Biology, Goldstein and Fyrberg, Eds., Academic Press, Inc., San Diego, Calif.). The preferred method of transposon mutagenesis in C. elegans employs Tc1 transposable element (Zwaal et al, supra; Plasterk et al., supra).

In addition to creating loss-of-function phenotypes, transposable elements can be used to incorporate the gene of interest, or mutant or derivative thereof, as an additional gene into any region of an animal's genome resulting in mis-expression (including over-expression) of the gene. A preferred vector designed specifically for misexpression of genes in transgenic Drosophila, is derived from pGMR (Hay et al., Development (1994) 120:2121-2129), is 9 Kb long, and contains: an origin of replication for E. coli; an ampicillin resistance gene; P element transposon 3′ and 5′ ends to mobilize the inserted sequences; a White marker gene; an expression unit comprising the TATA region of hsp70 enhancer and the 3′untranslated region of a-tubulin gene. The expression unit contains a first multiple cloning site (MCS) designed for insertion of an enhancer and a second MCS located 500 bases downstream, designed for the insertion of a gene of interest. As an alternative to transposable elements, homologous recombination or gene targeting techniques can be used to substitute a gene of interest for one or both copies of the animal's homologous gene. The transgene can be under the regulation of either an exogenous or an endogenous promoter element, and be inserted as either a minigene or a large genomic fragment. In one application, gene function can be analyzed by ectopic expression, using, for example, Drosophila (Brand et al., Methods in Cell Biology (1994) 44:635-654) or C. elegans (Mello and Fire, Methods in Cell Biology (1995) 48:451-482).

Examples of well-characterized heterologous promoters that may be used to create the transgenic animals include heat shock promoters/enhancers, which are useful for temperature induced mis-expression. In Drosophila, these include the hsp 70 and hsp83 genes, and in C. elegans, include hsp 16-2 and hsp 16-41. Tissue specific promoters/enhancers are also useful, and in Drosophila, include eyeless (Mozer and Benzer, Development (1994) 120:1049-1058), sevenless (Bowtell et al., PNAS (1991) 88(15):6853-6857), and glass-responsive promoters/enhancers (Quiring et al., Science (1994) 265:785-789) which are useful for expression in the eye; and enhancers/promoters derived from the dpp or vestigal genes which are useful for expression in the wing (Stachling-Hampton et al., Cell Growth Differ. (1994) 5(6):585-593; Kim et al., Nature (1996) 382:133-138). Finally, where it is necessary to restrict the activity of dominant active or dominant negative transgenes to regions where the pathway is normally active, it may be useful to use endogenous promoters of genes in the pathway, such as a subject protein pathway genes.

In C. elegans, examples of useful tissue specific promoters/enhancers include the myo-2 gene promoter, useful for pharyngeal muscle-specific expression; the hlh-1 gene promoter, useful for body-muscle-specific expression; and the gene promoter, useful for touch-neuron-specific gene expression. In a preferred embodiment, gene fusions for directing the mis-expression of a subject pathway gene are incorporated into a transformation vector which is injected into nematodes along with a plasmid containing a dominant selectable marker, such as rol-6. Transgenic animals are identified as those exhibiting a roller phenotype, and the transgenic animals are inspected for additional phenotypes of interest created by mis-expression of a subject pathway gene.

In Drosophila, binary control systems that employ exogenous DNA are useful when testing the mis-expression of genes in a wide variety of developmental stage-specific and tissue-specific patterns. Two examples of binary exogenous regulatory systems include the UAS/GAL4 system from yeast (Hay et al., PNAS (1997) 94(10):5195-5200; Ellis et al., Development (1993) 119(3):855-865); Brand and Perrimon (1993) Development 118(2):401-415), and the “Tet system” derived from E. coli (Bello et al., Development (1998) 125:2193-2202).

Dominant negative mutations, by which the mutation causes a protein to interfere with the normal function of a wild-type copy of the protein, and which can result in loss-of-function or reduced-function phenotypes in the presence of a normal copy of the gene, can be made using known methods (Hershkowitz, Nature (1987) 329:219-222).

Assays for Chance in Gene Expression

Various expression analysis techniques may be used to identify genes which are differentially expressed between a cell line or an animal expressing a wild type subject gene compared to another cell line or animal expressing a mutant subject gene. Such expression profiling techniques include differential display, serial analysis of gene expression (SAGE), transcript profiling coupled to a gene database query, nucleic acid array technology, subtractive hybridization, and proteome analysis (e.g. mass-spectrometry and two-dimensional protein gels). Nucleic acid array technology may be used to determine a global (i.e., genome-wide) gene expression pattern in a normal animal for comparison with an animal having a mutation in a subject gene. Gene expression profiling can also be used to identify other genes (or proteins) that may have a functional relation to a subject (e.g. may participate in a signaling pathway with a subject gene). The genes are identified by detecting changes in their expression levels following mutation, i.e., insertion, deletion or substitution in, or over-expression, under-expression, mis-expression or knock-out, of the dmHelicase gene.

Phenotypes Associated with Target Pathway Gene Mutations

After isolation of model animals carrying mutated or mis-expressed subject pathway genes or inhibitory RNAs, animals are carefully examined for phenotypes of interest. For analysis of subject pathway genes that have been mutated (i.e. deletions, insertions, and/or point mutations) animal models that are both homozygous and heterozygous for the altered subject pathway gene are analyzed. Examples of specific phenotypes that may be investigated include lethality; sterility; feeding behavior, perturbations in neuromuscular function including alterations in motility, and alterations in sensitivity to pesticides and pharmaceuticals. Some phenotypes more specific to flies include alterations in: adult behavior such as, flight ability, walking, grooming, phototaxis, mating or egg-laying; alterations in the responses of sensory organs, changes in the morphology, size or number of adult tissues such as, eyes, wings, legs, bristles, antennae, gut, fat body, gonads, and musculature; larval tissues such as mouth parts, cuticles, internal tissues or imaginal discs; or larval behavior such as feeding, molting, crawling, or puparian formation; or developmental defects in any germline or embryonic tissues. Some phenotypes more specific to nematodes include: locomotory, egg laying, chemosensation, male mating, and intestinal expulsion defects. In various cases, single phenotypes or a combination of specific phenotypes in model organisms might point to specific genes or a specific pathway of genes, which facilitate the cloning process.

Genomic sequences containing a subject pathway gene can be used to confirm whether an existing mutant insect or worm line corresponds to a mutation in one or more subject pathway genes, by rescuing the mutant phenotype. Briefly, a genomic fragment containing the subject pathway gene of interest and potential flanking regulatory regions can be subcloned into any appropriate insect (such as Drosophila) or worm (such as C. elegans) transformation vector, and injected into the animals. For Drosophila, an appropriate helper plasmid is used in the injections to supply transposase for transposon-based vectors. Resulting germline transformants are crossed for complementation testing to an existing or newly created panel of Drosophila or C. elegans lines whose mutations have been mapped to the vicinity of the gene of interest (Fly Pushing: The Theory and Practice of Drosophila Genetics, supra; and Caenorhabditis elegans: Modern Biological Analysis of an Organism (1995), Epstein and Shakes, eds.). If a mutant line is discovered to be rescued by this genomic fragment, as judged by complementation of the mutant phenotype, then the mutant line likely harbors a mutation in the subject pathway gene. This prediction can be further confirmed by sequencing the subject pathway gene from the mutant line to identify the lesion in the subject pathway gene.

Identification of Genes That Modify a Subject Genes

The characterization of new phenotypes created by mutations or misexpression in subject genes enables one to test for genetic interactions between subject genes and other genes that may participate in the same, related, or interacting genetic or biochemical pathway(s). Individual genes can be used as starting points in large-scale genetic modifier screens as described in more detail below. Alternatively, RNAi methods can be used to simulate loss-of-function mutations in the genes being analyzed. It is of particular interest to investigate whether there are any interactions of subject genes with other well-characterized genes, particularly genes involved in DNA unwinding.

Genetic Modifier Screens

A genetic modifier screen using invertebrate model organisms is a particularly preferred method for identifying genes that interact with subject genes, because large numbers of animals can be systematically screened making it more possible that interacting genes will be identified. In Drosophila, a screen of up to about 10,000 animals is considered to be a pilot-scale screen. Moderate-scale screens usually employ about 10,000 to about 50,000 flies, and large-scale screens employ greater than about 50,000 flies. In a genetic modifier screen, animals having a mutant phenotype due to a mutation in or misexpression of one or more subject genes are further mutagenized, for example by chemical mutagenesis or transposon mutagenesis.

The procedures involved in typical Drosophila genetic modifier screens are well-known in the art (Wolfner and Goldberg, Methods in Cell Biology (1994) 44:33-80; and Karim et al., Genetics (1996) 143:315-329). The procedures used differ depending upon the precise nature of the mutant allele being modified. If the mutant allele is genetically recessive, as is commonly the situation for a loss-of-function allele, then most typically males, or in some cases females, which carry one copy of the mutant allele are exposed to an effective mutagen, such as EMS, MMS, ENU, triethylamine, diepoxyalkanes, ICR-170, formaldehyde, X-rays, gamma rays, or ultraviolet radiation. The mutagenized animals are crossed to animals of the opposite sex that also carry the mutant allele to be modified. In the case where the mutant allele being modified is genetically dominant, as is commonly the situation for ectopically expressed genes, wild type males are mutagenized and crossed to females carrying the mutant allele to be modified.

The progeny of the mutagenized and crossed flies that exhibit either enhancement or suppression of the original phenotype are presumed to have mutations in other genes, called “modifier genes”, that participate in the same phenotype-generating pathway. These progeny are immediately crossed to adults containing balancer chromosomes and used as founders of a stable genetic line. In addition, progeny of the founder adult are retested under the original screening conditions to ensure stability and reproducibility of the phenotype. Additional secondary screens may be employed, as appropriate, to confirm the suitability of each new modifier mutant line for further analysis.

Standard techniques used for the mapping of modifiers that come from a genetic screen in Drosophila include meiotic mapping with visible or molecular genetic markers; male-specific recombination mapping relative to P-element insertions; complementation analysis with deficiencies, duplications, and lethal P-element insertions; and cytological analysis of chromosomal aberrations (Fly Pushing: Theory and Practice of Drosophila Genetics, supra; Drosophila: A Laboratory Handbook, supra). Genes corresponding to modifier mutations that fail to complement a lethal P-element may be cloned by plasmid rescue of the genomic sequence surrounding that P-element. Alternatively, modifier genes may be mapped by phenotype rescue and positional cloning (Sambrook et al., supra).

Newly identified modifier mutations can be tested directly for interaction with other genes of interest known to be involved or implicated with a subject gene using methods described above. Also, the new modifier mutations can be tested for interactions with genes in other pathways that are not believed to be related to neuronal signaling (e.g. nanos in Drosophila). New modifier mutations that exhibit specific genetic interactions with other genes implicated in neuronal signaling, but not interactions with genes in unrelated pathways, are of particular interest.

The modifier mutations may also be used to identify “complementation groups”. Two modifier mutations are considered to fall within the same complementation group if animals carrying both mutations in trans exhibit essentially the same phenotype as animals that are homozygous for each mutation individually and, generally are lethal when in trans to each other (Fly Pushing: The Theory and Practice of Drosophila Genetics, supra). Generally, individual complementation groups defined in this way correspond to individual genes.

When modifier genes are identified, homologous genes in other species can be isolated using procedures based on cross-hybridization with modifier gene DNA probes, PCR-based strategies with primer sequences derived from the modifier genes, and/or computer searches of sequence databases. For therapeutic applications related to the function of subject genes, human and rodent homologs of the modifier genes are of particular interest. For pesticide and other agricultural applications, homologs of modifier genes in insects and arachnids are of particular interest. Insects, arachnids, and other organisms of interest include, among others, Isopoda; Diplopoda; Chilopoda; Symphyla; Thysanura; Collembola; Orthoptera, such as Scistocerca spp; Blattoidea, such as Blattella germanica; Dermaptera; Isoptera; Anoplura; Mallophaga; Thysanoptera; Heteroptera; Homoptera, including Bemisia tabaci, and Myzus spp.; Lepidoptera including Plodia interpunctella, Pectinophora gossypiella, Plutella spp., Heliothis spp., and Spodoptera species; Coleoptera such as Leptinotarsa, Diabrotica spp., Anthonomus spp., and Tribolium spp.; Hymenoptera; Diptera, including Anopheles spp.; Siphonaptera, including Ctenocephalides felis; Arachnida; and Acarinan, including Amblyoma americanum; and nematodes, including Meloidogyne spp., and Heterodera glycinii.

Although the above-described Drosophila genetic modifier screens are quite powerful and sensitive, some genes that interact with subject genes may be missed in this approach, particularly if there is functional redundancy of those genes. This is because the vast majority of the mutations generated in the standard mutagenesis methods will be loss-of-function mutations, whereas gain-of-function mutations that could reveal genes with functional redundancy will be relatively rare. Another method of genetic screening in Drosophila has been developed that focuses specifically on systematic gain-of-function genetic screens (Rorth et al., Development (1998) 125:1049-1057). This method is based on a modular mis-expression system utilizing components of the GAL4/UAS system (described above) where a modified P element, termed an “enhanced P” (EP) element, is genetically engineered to contain a GAL4-responsive UAS element and promoter. Any other transposons can also be used for this system. The resulting transposon is used to randomly tag genes by insertional mutagenesis (similar to the method of P element mutagenesis described above). Thousands of transgenic Drosophila strains, termed EP lines, can be generated, each containing a specific UAS-tagged gene. This approach takes advantage of the preference of P elements to insert at the 5′-ends of genes. Consequently, many of the genes that are tagged by insertion of EP elements become operably fused to a GAL4-regulated promoter, and increased expression or mis-expression of the randomly tagged gene can be induced by crossing in a GAL4 driver gene.

Systematic gain-of-function genetic screens for modifiers of phenotypes induced by mutation or mis-expression of a subject gene can be performed by crossing several thousand Drosophila EP lines individually into a genetic background containing a mutant or mis-expressed subject gene, and further containing an appropriate GAL4 driver transgene. It is also possible to remobilize the EP elements to obtain novel insertions. The progeny of these crosses are then analyzed for enhancement or suppression of the original mutant phenotype as described above. Those identified as having mutations that interact with the subject gene can be tested further to verity the reproducibility and specificity of this genetic interaction. EP insertions that demonstrate a specific genetic interaction with a mutant or mis-expressed subject gene, have a physically tagged new gene which can be identified and sequenced using PCR or hybridization screening methods, allowing the isolation of the genomic DNA adjacent to the position of the EP element insertion.

### EXAMPLES

The following examples describe the isolation and cloning of the nucleic acid sequence of SEQ ID NOS:1, 3, and 5 and how these sequences, and derivatives and fragments thereof, as well as other pathway nucleic acids and gene products can be used for genetic studies to elucidate mechanisms of a pathway involving a subject protein as well as the discovery of potential pharmaceutical or pesticidal agents that interact with the pathway.

These Examples are provided merely as illustrative of various aspects of the invention and should not be construed to limit the invention in any way.

### Example 1

### Preparation of Drosophila cDNA Library

A Drosophila expressed sequence tag (EST) cDNA library was prepared as follows. Tissue from mixed stage embryos (0-20 hour), imaginal disks and adult fly heads were collected and total RNA was prepared. Mitochondrial rRNA was removed from the total RNA by hybridization with biotinylated rRNA specific oligonucleotides and the resulting RNA was selected for polyadenylated mRNA. The resulting material was then used to construct a random primed library. First strand cDNA synthesis was primed using a six nucleotide random primer. The first strand cDNA was then tailed with terminal transferase to add approximately 15 dGTP molecules. The second strand was primed using a primer which contained a Not1 site followed by a 13 nucleotide C-tail to hybridize to the G-tailed first strand cDNA. The double stranded cDNA was ligated with BstX1 adaptors and digested with Not1. The cDNA was then fractionated by size by electrophoresis on an agarose gel and the cDNA greater than 700 bp was purified. The cDNA was ligated with Not1, BstX1 digested pcDNA-sk+vector (a derivative of pBluescript, Stratagene) and used to transform E. coli (XL1blue). The final complexity of the library was 6×10⁶ independent clones.

The cDNA library was normalized using a modification of the method described by Bonaldo et al. (Genome Research (1996) 6:791-806). Biotinylated driver was prepared from the cDNA by PCR amplification of the inserts and allowed to hybridize with single stranded plasmids of the same library. The resulting double-stranded forms were removed using strep avidin magnetic beads, the remaining single stranded plasmids were converted to double stranded molecules using Sequenase (Amersham, Arlington Hills, Ill.), and the plasmid DNA stored at −20° C. prior to transformation. Aliquots of the normalized plasmid library were used to transform E. coli (XL1blue or DH10B), plated at moderate density, and the colonies picked into a 384-well master plate containing bacterial growth media using a Qbot robot (Genetix, Christchurch, UK). The clones were allowed to grow for 24 hours at 37° C. then the master plates were frozen at −80° C. for storage. The total number of colonies picked for sequencing from the normalized library was 240,000. The master plates were used to inoculate media for growth and preparation of DNA for use as template in sequencing reactions. The reactions were primarily carried out with primer that initiated at the 5′ end of the cDNA inserts. However, a minor percentage of the clones were also sequenced from the 3′ end. Clones were selected for 3′ end sequencing based on either further biological interest or the selection of clones that could extend assemblies of contiguous sequences (“contigs”) as discussed below. DNA sequencing was carried out using AB1377 automated sequencers and used either ABI FS, dirhodamine or BigDye chemistries (Applied Biosystems, Inc., Foster City, Calif.).

Analysis of sequences were done as follows: the traces generated by the automated sequencers were base-called using the program “Phred” (Gordon, Genome Res. (1998) 8:195-202), which also assigned quality values to each base. The resulting sequences were trimmed for quality in view of the assigned scores. Vector sequences were also removed. Each sequence was compared to all other fly EST sequences using the BLAST program and a filter to identify regions of near 100% identity. Sequences with potential overlap were then assembled into contigs using the programs “Phrap”, “Phred” and “Consed” (Phil Green, University of Washington, Seattle, Wash.; http://bozeman.mbt.washington.edu/phrap.docs/phrap.html). The resulting assemblies were then compared to existing public databases and homology to known proteins was then used to direct translation of the consensus sequence. Where no BLAST homology was available, the statistically most likely translation based on codon and hexanucleotide preference was used. The Pfam (Bateman et al, Nucleic Acids Res. (1999) 27:260-262) and Prosite (Hoffmann et al., Nucleic Acids Res. (1999) 27(1):215-219) collections of protein domains were used to identify motifs in the resulting translations. The contig sequences were archived in an Oracle-based relational database (FlyTag™, Exelixis, Inc., South San Francisco, Calif.)

### Example 2

### Discovery of Novel Targets from a P-Lethal Screen

dmHelicase was discovered from a screen using collections of P element transposon-induced recessive lethal mutations (P lethals) to identify novel genes. Briefly, genomic sequence surrounding transposable element 1(3)06945, (http://www.fruitflv.org/cgi-bin/bfd/bfd_namesearch.p1?caller_class=form&types=Insertion&clue=1%283%2906945&cs=&cc=) was retrieved by inverse PCR, and blasted against the FlyTag™ database, which resulted in identification of pertinent clones for full-length cloning.

dmPITP was discovered from a screen using collections of P element transposon induced recessive lethal mutations (P lethals) to identify novel genes. Briefly, genomic sequence surrounding transposable element EP(3)0513 (GI3738449: 3prime Drosophila melanogaster EP line Drosophila melanogaster genomic Sequence recovered from 3′ end of P element, genomic survey sequence) was retrieved by inverse PCR, and BLASTed against the FlyTag™ database, which resulted in identification of pertinent clones for full-length cloning.

dmSPL1 was discovered from a screen using collections of P element transposon induced recessive lethal mutations (P lethals) to identify novel genes. Briefly, genomic sequence surrounding transposable element 1(2)05091 (http://www.fruitfly.org/cgi-bin/bfd/transposon_report.p1?transposon=1(2)05091) was retrieved by inverse PCR, and BLASTed against the FlyTag™ database, which resulted in identification of pertinent clones for full-length cloning.

### Example 3

### Cloning of Subject Nucleic Acid Sequences

Unless otherwise noted, the PCR conditions used for cloning the nucleic acid sequences set forth in SEQ ID NOS:1, 3, and 5 was as follows: A denaturation step of 94° C., 5 min; followed by 35 cycles of: 94° C. 1 min, 55° C. 1 min 72° C. 1 min; then, a final extension at 72° C. 10 min.

All DNA sequencing reactions were performed using standard protocols for the BigDye sequencing reagents (Applied Biosystems, Inc.) and products were analyzed using ABI 377 DNA sequencers. Trace data obtained from the ABI 377 DNA sequencers was analyzed and assembled into contigs using the Phred-Phrap programs.

Well-separated, single colonies were streaked on a plate and end-sequenced to verify the clones. Single colonies were picked and the enclosed plasmid DNA was purified using Qiagen REAL Preps (Qiagen, Inc., Valencia, Calif.). Samples were then digested with appropriate enzymes to excise insert from vector and determine size, for example the vector pOT2, (www.fruitfly.org/EST/pOT2vector.html) and can be excised with XhoI/EcoRI; or pBluescript (Stratagene) and can be excised with BssH II. Clones were then sequenced using a combination of primer walking and in vitro transposon tagging strategies.

For primer walking, primers were designed to the known DNA sequences in the clones, using the Primer-3 software (Steve Rozen, Helen J. Skaletsky (1998) Primer3. Code available at http://www-genome.wi.mit.edu/genome_software/other/primer3.html.). These primers were then used in sequencing reactions to extend the sequence until the full sequence of the insert was determined.

The GPS-1 Genome Priming System in vitro transposon kit (New England Biolabs, Inc., Beverly, Mass.) was used for transposon-based sequencing, following manufacturer's protocols. Briefly, multiple DNA templates with randomly interspersed primer-binding sites were generated. These clones were prepared by picking 24 colonies/clone into a Qiagen REAL Prep to purify DNA and sequenced by using supplied primers to perform bidirectional sequencing from both ends of transposon insertion.

Sequences were then assembled using Phred/Phrap and analyzed using Consed. Ambiguities in the sequence were resolved by resequencing several clones. This effort resulted in identification of various nucleic acid molecules, which are described in detail below.

dmHelicase

A dmHelicase nucleic acid molecule was identified in a contiguous nucleotide sequence of 1776 bases in length, encompassing an open reading frame (ORF) of 1443 nucleotides encoding a predicted protein of 481 amino acids. The ORF extends from base 162-1604 of SEQ ID NO: 1.

dmPITP

A dmPITP nucleic acid molecule was identified in a contiguous nucleotide sequence of 1066 bases in length, encompassing an open reading frame (ORF) of 816 nucleotides encoding a predicted protein of 272 amino acids. The ORF extends from base 183-998 of SEQ ID NO:3.

dmSPL

A dmSPL nucleic acid molecule was identified in a contiguous nucleotide sequence of 2060 bases in length, encompassing an open reading frame (ORF) of 1635 nucleotides encoding a predicted protein of 545 amino acids. The ORF extends from base 110-1744 of SEQ ID NO:5.

### Example 4

### Analysis of Identified Nucleic Acid Sequences

Upon completion of cloning described above, the sequences were analyzed using the Pfam and Prosite programs.

dmHelicase

Pfam recognized ATPase domain associated with various cellular activities (PF00004) at amino acids 68-411 of SEQ ID NO:2, corresponding to nucleotides 366-1395 of SEQ ID NO: 1. Prosite recognized several putative motifs, which are summarized in Table 1:

TABLE 1

| | | AMINO ACID | NUCLEOTIDE |
|---|---|---|---|
| MOTIF | PROSITE # | RESIDUES | RESIDUES |
| N-Glycosylation site | PDOC00001; | 433-436 | 1461-1470 |
| | PS00001 | | |
| CAMP and cGMP | PDOC00004; | 412-415 | 1398-1407 |
| dependent protein | PS00004 | | |
| kinase phosphorylation | | | |
| site | | | |
| Protein Kinase C | PDOC00005; | 4-6 | 174-180 |
| phosphorylation site | PS00005 | 77-79 | 393-399 |
| | | 158-160 | 636-642 |
| | | 195-197 | 747-753 |
| | | 324-326 | 1134-1140 |
| | | 359-361 | 1239-1245 |
| | | 394-396 | 1344-1350 |
| | | 435-437 | 1467-1473 |
| Casein Kinase II | PDOC00006; | 13-16 | 201-210 |
| Phosphorylation site | P500006 | 100-103 | 462-471 |
| | | 110-113 | 492-501 |
| | | 162-165 | 648-657 |
| | | 166-169 | 660-669 |
| | | 239-242 | 879-888 |
| | | 359-362 | 1239-1248 |
| N-Myristolation site | PDOC00008; | 44-49 | 294-309 |
| | PS00008 | 85-90 | 417-432 |
| | | 151-156 | 615-630 |
| | | 472-477 | 1578-1593 |
| ATP/GTP binding site | PDOC00017; | 73-80 | 380-401 |
| motif A | PS00017 | | |

Nucleotide and amino acid sequences for the dmHelicase nucleic acid sequence and its encoded protein were searched against all available nucleotide and amino acid sequences in the public databases, using BLAST (Altschul et al., supra). Table 2 below summarizes the results. The 5 most similar sequences are listed.

TABLE 2

| GI # | DESCRIPTION |
|---|---|
| DNA BLAST | |
| 6436109 = AC015226 | Drosophila melanogaster, *** SEQUENCING |
| | IN PROGRESS ***, in ordered pieces |
| 5609255 = AL097644 | Drosophila melanogaster genome survey |
| | sequence SP6 end of BAC BACN02G08 |
| | of DrosBAC library from Drosophila |
| | melanogaster (fruit fly), genomic |
| | survey sequence |
| 5670650 = AC006497 | Drosophila melanogaster chromosome |
| | 3 clone BACR48B15 (D548) RPCI-98 48.B.15 |
| | map 76A3-B4 strain y; cn bw sp, *** |
| | SEQUENCING IN PROGRESS***, 82 |
| | unordered pieces. |
| 2795508 = AA540640 | LD20394.5prime LD Drosophila melanogaster |
| | embryo BlueScript Drosophila melanogaster |
| | cDNA clone LD20394 5prime, mRNA sequence |
| 4587310 = AB024301 | Homo sapiens mRNA for RuvB-like |
| | DNA helicase TIP49b, complete cds |
| PROTEIN BLAST | |
| 4587311 = BAA76708 | (AB024301) RuvB-like DNA helicase |
| | TIP49b [Homo sapiens] |
| 5020422 = AAD38073 | (AF155138) RUVBL2 protein |
| | [Homo sapiens] |
| 5326998 = CAB46270 | (Y18417) erythrocyte cytosolic protein |
| | of 51 kDa, ECP-51 [Homo sapiens] |
| 4521249 = BAA76297 | (AB013912) DNA helicase [Mus musculus] |
| 4929561 = AAD34041 | (AF151804) CGI-46 protein [Homo sapiens] |

The closest homolog predicted by BLAST analysis is a RuvB-like DNA helicase TIP49b from humans, sharing 78% identity and 90% homology with dmHelicase. TIP49a and TIP49b are both mammalian homologs of bacterial RuvB, and are found in the same ˜700 kDa complex in the cell. TIP49a and TIP49b share similar enzymatic properties and have ATPase activity; however, the polarity of TIP49b's helicase activity (5′ to 3′; same as RuvB) is reversed relative to TIP49a. Both TIP49a and TIP49b have been shown to be independently essential for cell growth, suggesting that their activities are not complementary.

While dmHelicase is clearly a DNA-helicase of the RuvB type with strong sequence identity to TIP49b, it is not clear that this is the eukaryotic orthologue of bacterial RuvB. There is closer homology amongst the eukaryotic TIP49s and dmHelicase (60-90%), than there is to the bacterial RuvB's (27%). Closer homology to eukaryotic sequences might suggest that either eukaryotic RuvB-type helicases diverged very early in evolution, and have since evolved at similar rates. Alternatively, it might be that the TIP49s and dmHelicase may form an as yet unidentified sub-family of RuvB-like helicases with variance in specificity.

BLAST results for the dmHelicase amino acid sequence indicate 24 amino acid residues as the shortest stretch of contiguous amino acids that is novel with respect to prior art sequences and 49 amino acids as the shortest stretch of contiguous amino acids for which there are no sequences contained within public database sharing 100% sequence similarity.

dmPITP

Prosite predicted the following putative motifs: Protein tyrosine kinase phosphorylation sites at amino acid residues 63-65, 170-172, 173-175, 217-219, and 233-235 (nucleotides 371-377, 692-698, 701-707, 833-839, and 881-887); Casein kinase II phosphorylation sites at amino acid residues 13-16, 24-27, 208-211, 240-243, and 251-254 (nucleotides 221-230, 254-263, 806-815, 902-911, 935-944); tyrosine kinase phosphorylation site at amino acids 160-168 (nucleotides 662-686); and N-myristolation sites at amino acids 34-39, and 54-59 (nucleotides 284-299, and 344-359).

Nucleotide and amino acid sequences of the dmPITP nucleic acid sequence and its encoded protein were searched against all available nucleotide and amino acid sequences in the public databases, using BLAST (Altschul et al., supra). Table 3 below summarizes the results. The 5 most similar sequences are listed.

TABLE 3

| GI # | DESCRIPTION |
|---|---|
| DNA BLAST | |
| 4201917 = AI387906 | Drosophila melanogaster cDNA clone GH18602 |
| | 5prime, mRNA sequence |
| 4444939 = AI530804 | Drosophila melanogaster cDNA clone SD01527 |
| | 5prime, mRNA |
| 4544354 = AC006091 | Drosophila melanogaster chromosome 3 clone |
| | BACR48G05 (D475) RPCI-98 48.G.5 map |
| | 91F1-91F13 strain y2; cn bw sp, |
| | *** SEQUENCING IN PROGRESS ***, |
| | 2 unordered pieces. |
| 5630022 = AC006091 | Drosophila melanogaster chromosome 3 clone |
| | BACR48G05 (D475) RPCI-98 48.G.5 map |
| | 91F1-91F13 strain y; cn bw sp, |
| | *** SEQUENCING IN PROGRESS ***, |
| | 4 unordered pieces. |
| 2701176 = AA698247 | Drosophila melanogaster cDNA clone HL04023 |
| | 5prime, mRNA sequence |
| PROTEIN BLAST | |
| 1060905 = BAA06277 | phosphatidylinositol transfer protein |
| | [Homo sapiens] |
| 628018 = JX0316 | phosphatidylinositol transfer protein |
| | beta isoform - rat |
| 829055 = BAA04669 | phosphatidylinositol transfer protein |
| | [Rattus norvegicus] |
| 1184995 = AAA87593 | phosphatidylinositol transfer protein |
| | beta isoform [Mus musculus] |
| 534829 = AAB08971 | phosphatidylinositol transfer protein |
| | [Oryctolagus cuniculus] |

The dmPITP gene and protein disclosed here is the first PITP described outside of mammalian cells. The closest homolog predicted by BLAST analysis is a human phosphatidyl transfer protein, sharing 64% identity and 77% similarity with dmPITP.

The BLAST analysis also revealed several other PITP proteins which share significant amino acid homology with dmPITP. The dmPITP is difficult to classify on the basis of primary sequence identity alone. The mammalian alpha and beta isoforms are quite distinct, sharing only 77% identity in human, while the alpha isoform is 97-98% identical between human and rabbit, mouse and rat. However, dmPITP is 59% identical with human PITP-A and 64% identical with human PITP-β. The areas of greatest sequence deviation involve charge reversals in the 110-130 region, an insertion between 50-60, loss of a charge at 160 and an excision at 190. Phylogenetically, dmPITP is perhaps more closely related to the beta isoforms, but is nearly equally distal from both sub-families. One means of classifying this protein may be to profile its lipid binding propensities. The capability to bind sphingomyelin in addition to PI and PC would identify this as more similar to PITP-β and exclude it from the PITP-α sub-family.

BLAST results for the dmPITP amino acid sequence indicate 14 amino acid residues as the shortest stretch of contiguous amino acids that is novel with respect to prior art sequences and 27 amino acids as the shortest stretch of contiguous amino acids for which there are no sequences contained within public database sharing 100% sequence similarity.

dmSPL

The predicted domains include: a transmembrane domain at amino acids 300-316 (nucleotides 1009-1057); a pyridoxal dependent decarboxylase conserved domain (PF 00282) at amino acids 192-306 (nucleotides 685-1027); a cystein/methionin metabolism PLP dependent enzyme domain (PF01053) at amino acids 133-431 (nucleotides 508-1402); and a DegT, DnrJ, EryC 1, StrS family (PF01041) at amino acids 138-522 (nucleotides 523-1675).

Nucleotide and amino acid sequences for the dmSPL1 nucleic acid sequences and their encoded proteins were searched against all available nucleotide and amino acid sequences in the public databases, using BLAST (Altschul et al., supra). Table 4 below summarizes the results. The 5 most similar sequences are listed.

TABLE 4

| GI # | DESCRIPTION |
|---|---|
| DNA BLAST | |
| 5670603 = AC007520 | Drosophila melanogaster chromosome 2 clone |
| | BACR11M15 (D609) RPCI-98 11.M.15 map |
| | 53D-54A strain y; cn bw sp, *** SEQUENCING |
| | IN PROGRESS ***, 88 unordered pieces |
| 4803905 = AC007520 | Drosophila melanogaster chromosome 2 clone |
| | BACR11M15 (D609) RPCI-98 11.M.15 map |
| | 53D-54A strain y2; cn bw sp, |
| | *** SEQUENCING IN PROGRESS***, |
| | 22 unordered pieces. |
| 3945967 = AI296560 | Drosophila melanogaster cDNA clone LP10512 |
| | 5prime, mRNA sequence |
| 4445868 = AI531733 | Drosophila melanogaster cDNA clone SD02978 |
| | 5prime, mRNA sequence |
| 4448308 = AI534173 | Drosophila melanogaster cDNA clone SD06695 |
| | 5prime, MRNA sequence |
| PROTEIN BLAST | |
| 2906011 = AAC03768 | Sphingosine-1-phosphate lyase; |
| | pyridoxal-phosphate protein; SPL |
| | [Mus musculus] |
| 6330874 = BAA86566 | KIAA1252 protein [Homo sapiens] |
| 4160532 = CAA09590 | Sphingosine-1-phosphate lyase |
| | [Homo sapiens] |
| No GI #. Published | Sphingosine-1-phosphate lyase, |
| PCT application | C. elegans |
| WO 9916888-A2, | |
| claim 11 | |
| No GI #. Published | Sphingosine-1-phosphate lyase, |
| PCT application | yeast |
| WO 9916888-A2, | |
| claim 11 | |

The closest homolog predicted by BLAST analysis is a sphingosine phosphate lyase from mouse, with 49% identity and 69% similarity with dmSPL1.

The BLAST analysis also revealed several other proteins that share significant amino acid homology with dmSPL1.

BLAST results for the dmSPL1 amino acid sequence indicate 15 amino acid residues as the shortest stretch of contiguous amino acids that is novel with respect to prior art sequences and 36 amino acids as the shortest stretch of contiguous amino acids for which there are no sequences contained within public database sharing 100% sequence similarity.

### Example 5

### Assays for ATP Hydrolysis

ATPase activity is assayed by use of activated charcoal (Sigma, St Louis, Mich.) as described previously (Armon et al., J. Biol. Chem. (1990) 265:20723-20726). The reaction (20 μl) contains 0.3 μg of the purified dmHelicase, unless specified otherwise. The dmHelicase is incubated at 37° C. for 30 min A buffer (20 mM Tris/HCl (pH 7.5), 70 mM KCl, 2.5 mM MgCl₂, 1.5 mM dithiothreitol, 0.1 mM ATP, and 1.25 mCi of [γ32P]ATP). One microgram of M13 single-stranded DNA (ssDNA), double-stranded pBluescript DNA (Stratagene, LaJolla, Calif.), RNA homopolymers (Amersham Pharmacia Biotech), or cellular total RNA is added to each reaction. Radioactivity is determined as Cerenkov radiation. Control reactions without dmHelicase are carried out in parallel tubes, and the control value (radioactivity) is subtracted from each experimental one. Each assay is done in duplicate, and the results are presented as a simple arithmetic average.

### Example 6

### DNA Helicase Assay

A complementary oligonucleotide corresponding to nucleotide positions 6291-6320 in M13 mp 18 ssDNA is synthesized and labeled at the 5′-end by T4 polynucleotide kinase and [γ-32P]ATP. The labeled oligonucleotide is annealed with the phage ssDNA by incubation at 95° C. for 10 min and 60 min at 37° C. The product is purified to remove the unannealed oligonucleotide. A complementary oligonucleotide (54-mer) including the SmaI site, corresponding to nucleotide positions 6226-6279 in M13 mp 18 ssDNA, is synthesized and hybridized with the phage ssDNA. The oligonucleotide is labeled with T4 DNA kinase for 5′-end labeling or with terminal deoxynucleotidyl transferase and [γ-32P]ddATP for 3′-end labeling. After SmaI digestion, this partial duplex DNA is used as a substrate.

For the DNA helicase assay, the reaction mixture (20 μl) contains 20 mM Tris/HCl (pH 7.5), 2 mM dithiothreitol, 50 mg/ml BSA, 0.5 mM MgCl₂, 80 mM KCl, 1 mM ATP, and 10 ng of 32P-labeled helicase substrate. The reactions also contain 0.2 μg of the purified dmHelicase. Compounds that might modulate the helicase activity may also be added as competitiors (0.2 μg). The helicase assay is performed at 37° C. for 30 min and stopped by the addition of 5 ml of 60 mM EDTA, 0.75% SDS, and 0.1% bromphenol blue. The reaction mixture is then subjected to 10% PAGE, and the displaced oligonucleotides are visualized by autoradiography.

### Example 7

### Purification of dmPITP

Clones containing dmPITP sequence are subcloned into the BamHI-SalI restriction sites of the pBluescript vector and transformed into XL 1-Blue cells (Stratagene, La Jolla, Calif.). Positive clones are resequenced to verify the correct clones. Inserts are then subcloned into the expression vector pET21 a to generate the dmPITP-hexahistidine fusion construct and transformed into BL21(DE3) cells (Novagen, Madison, Wis.). DmPITP is induced with isopropyl b-D-thiogalactoside (IPTG; 0.1 mM) for 4 hr at room temperature and bacterial cells are collected by centrifugation. The pellet is resuspended in buffer containing 50 mM sodium phosphate and 300 mM NaCl (pH 8.0). Lysozyme (1 mg/ml) is then added and incubated at 4° C. for 30 min. The sample is then sonicated 6×1 min on ice and centrifuged at 10,000×g for 30 min at 4° C. The supernatant is mixed with Ni²⁺-NTA agarose resin (Qiagen, Valencia, Calif.) (4 ml of a 50% NTA slurry) for 30 min at 4° C. and then transferred to a prepared column. The column is washed with 12 bed volumes with buffer containing 50 mM sodium phosphate, 300 mM NaCl, and 10% glycerol at pH 6.0 (wash buffer), followed by 6 bed volumes of wash buffer but containing 525 mM NaCl and 6 bed volumes containing 525 mM NaCl and 25 mM imidazole. Protein is then eluted with 1.5 bed volumes of wash buffer containing 525 mM NaCl and 250 mM imidazole dmPITP is then exchanged into 20 mM Pipes, 137 mM NaCl, 3 mM KCl (pH 6.8), and loaded onto Superdex-75 (Pharmacia, Kalamazoo, Mich.)). Active fractions (assayed by in vitro PI transfer activity) are pooled and concentrated.

### Example 8

### Assays for Phosphatidylinositol (PI) and Phosphatidylcholine (PC) Transfer

PI transfer activity is assayed as described previously (Thomas et al., supra). This assay measures the transfer of [³H]-PI from rat liver microsomes to unlabeled liposomes in the presence of transfer protein dmPITP). Protein samples of dmPITP are added to tubes containing [³H]PI-labeled microsomes (62.5 μg of microsome protein), liposomes (50 mmol of phospholipid; 98 mol % PC:2 mol % PI), and SET buffer (0.25 M sucrose, 1 mM EDTA, and 5 mM Tris-HCl (pH 7.4)) in a final volume of 125 μl. Pharmaceutical or insecticidal compounds may be added along with dmPITP at this stage. After incubation at 27° C. for 30 minutes, microsomes are precipitated by the addition of 25 μl of ice-cold 0.2 M sodium acetate (pH 5.0) and removed by centrifugation (12,000×g for 15 min). A 100-μl aliquot of the supernatant is measured for radioactivity.

Assay for PC transfer activity measures the transfer of radioactivity from [³C]PC-labeled liposomes to rat liver mitochondria. The liposomes consist of 2 mmol of egg yolk PC/ml containing 1 μCi of [³H]PC in SET buffer and are sonicated on ice prior to use. [³H]PC-labeled liposomes (40 mmol) are incubated with dmPITP (in presence or absence of compounds) and rat liver mitochondria (2 mg of protein) in a final volume of 0.2 ml of SET buffer for 30 min at 37° C. The reactions are halted by placing samples on ice, and mitochondria are sedimented by centrifugation at 12,000×g for 10 min. The sedimented mitochondria are resuspended in 0.5 ml of SET buffer and sedimented by centrifugation at 12,000×g for 10 min through 0.5 ml of 14.3% sucrose. The pellet is resuspended in 50 μl of 10% SDS and boiled for 5 min, and this solution is counted for radioactivity.

### Example 9

### Sphingosine-Phosphate Lyase Assay

Lyase activity is measured by following the formation of labeled fatty aldehyde (and further metabolites) from [³H]dihydrosphingosine-phosphate. Assays are performed in glass tubes (13×100 mm) as follows. An aliquot of [³H]dihydrosphingosine—phosphate (10 mmol), dissolved in methanol, is placed in a tube and dried under N₂. To dissolve this material, 25 μL of 1% (w/v) Triton X-100 is added, followed by 175 μL of reaction mixture. In order to ensure complete dissolution of the lipid, tubes are placed in a bath sonicator for 30 sec. Reactions are started by adding 50 μL of sample, in presence or absence of compounds, diluted in a homogenization medium. Standard final concentrations are: 50 mM sucrose, 100 mM K-phosphate buffer pH 7.4, 25 mM NaF, 0.1% (w/v) Triton X-100, 0.5 mM EDTA, 2 mM DTT, 0.25 mM pyridoxal phosphate, 40 μM dihydrosphingosine-phosphate. After 1 hr of incubation at 37° C., reactions are terminated by adding 0.3 mL of 1% (W/V) HClO₄, followed by 2.1 mL of chloroform/methanol (1/2—v/v). After vortexing, phase separation is induced by adding 0.7 ml of 1% (w/v) HClO₄ and 0.7 ml of chlorofom. Tubes are again vortexed and centrifuged. The upper phase is removed and the lower phase is washed twice with 1.4 mL of 1% (w/v) HClO₄/methanol (8/2—v/v). An aliquot of the lower phase (1 mL/1.25 mL total) is transferred to another tube, dried under N₂, and dissolved in 50-100 μL of chloroform, containing palmitic acid, palmitol and palmitaldehyde, each 5 mM final concentration. Aliquots (20 μL) are spotted on silica 60 G plates (Merck, Rahway, N.J.) and developed in solvent F (hexane/diethyl ether/acetic acid 70/29/1 v/v) and/or G (chloroform/methanol/acetic acid 50/49/1 v/v). The first system is used if separation of the fatty aldehyde metabolites is required. After development, plates are allowed to dry and exposed to iodine fumes. Selective staining for aldehyde is also performed. Regions of interest are scraped into scintillation vials containing 1 mL of 1% (w/v) SDS. Before counting, 8 mL of Instagel II (Canberra-Packard, Meriden, Conn., USA) is added to the vials. When separation of the metabolites is not needed, solvent G is employed. In this more polar solvent, all metabolites run close together near the front. In this case the whole region is scraped into vials and counted.

What is claimed is:

1. An isolated nucleic acid molecule of less than about 15 kb in size comprising a nucleic acid sequence that encodes an invertebrate receptor polypeptide and that shares at least about 75% nucleotide sequence identity with the sequence set forth in SEQ ID NO:1, or the complement thereof.

2. An isolated nucleic acid molecule of less than about 15 kb in size comprising a nucleic acid sequence that encodes an invertebrate receptor polypeptide and that shares at least about 75% nucleotide sequence identity with the sequence set forth in SEQ ID NO:3, or the complement thereof.

3. An isolated nucleic acid molecule of less than about 15 kb in size comprising a nucleic acid sequence that encodes an invertebrate receptor polypeptide and that shares at least about 75% nucleotide sequence identity with the sequence set forth in SEQ ID NO:5, or the complement thereof.

4. An isolated nucleic acid molecule comprising a nucleic acid sequence that encodes a polypeptide comprising at least 36 amino acids that share 100% sequence identity with 36 contiguous amino acids of SEQ ID NO:2.

5. The isolated nucleic acid molecule of claim 4 wherein said nucleic acid sequence encodes the entire sequence of SEQ ID NO:2.

6. The isolated nucleic acid molecule of claim 4 wherein said nucleic acid sequence encodes a polypeptide having helicase activity.

7. An isolated nucleic acid molecule comprising a nucleic acid sequence that encodes a polypeptide comprising at least 27 amino acids that share 100% sequence identity with 27 contiguous amino acids of SEQ ID NO:4.

8. The isolated nucleic acid molecule of claim 7 wherein said nucleic acid sequence encodes the entire sequence of SEQ ID NO:4.

9. The isolated nucleic acid molecule of claim 7 wherein said nucleic acid sequence encodes a protein having phospholipid transfer activity.

10. An isolated nucleic acid molecule comprising a nucleic acid sequence that encodes a polypeptide comprising at least 46 amino acids that share 100% sequence identity with 46 contiguous amino acids of SEQ ID NO:6.

11. The isolated nucleic acid molecule of claim 10 wherein said nucleic acid sequence encodes the entire sequence of SEQ ID NO:6.

12. The isolated nucleic acid molecule of claim 10 wherein said nucleic acid sequence encodes a protein having sphingosine phosphate lyase activity.

13. A vector comprising the nucleic acid molecule of any one of claims 1, 4, 5, or 6.

14. A host cell comprising the vector of claim 13.

15. A vector comprising the nucleic acid molecule of any one of claims 2, 7, 8, or 9.

16. A host cell comprising the vector of claim 15.

17. A vector comprising the nucleic acid molecule of any one of claims 3, 10, 11, or 12.

18. A host cell comprising the vector of claim 17.

19. A process for producing an invertebrate helicase protein comprising culturing the host cell of claim 14 under conditions suitable for expression of said helicase protein and recovering said protein.

20. A process for producing an invertebrate phosphatidylinositol transfer protein (PITP) comprising culturing the host cell of claim 16 under conditions suitable for expression of said PITP and recovering said protein.

21. A process for producing an invertebrate sphingosine phosphate lyase (SPL) comprising culturing the host cell of claim 18 under conditions suitable for expression of said SPL and recovering said protein.

22. A purified protein comprising an amino acid sequence having at least about 80% sequence identity with any one of the sequences set forth in SEQ ID NOS:2, 4, or 6.

23. A method for detecting a candidate compound that interacts with a helicase protein or fragment thereof, said method comprising contacting said helicase protein or fragment with one or more candidate molecules, and detecting any interaction between said candidate compound and said helicase protein or fragment; wherein the amino acid sequence of said helicase protein comprises an amino acid sequence which is at least about 80% identical to the sequence set forth in SEQ ID NO:2.

24. A method for detecting a candidate compound that interacts with a phosphatidylinositol transfer protein (PITP) or fragment thereof, said method comprising contacting said PITP or fragment with one or more candidate molecules, and detecting any interaction between said candidate compound and said PITP or fragment; wherein the amino acid sequence of said PITP comprises an amino acid sequence which is at least about 80% identical to the sequence set forth in SEQ ID NO:4.

25. A method for detecting a candidate compound that interacts with a sphingosine phosphate lyase (SPL) or fragment thereof, said method comprising contacting said SPL or fragment with one or more candidate molecules, and detecting any interaction between said candidate compound and said SPL or fragment; wherein the amino acid sequence of said SPL protein comprises an amino acid sequence which is at least about 80% identical to the sequence set forth in SEQ ID NO:6.

26. The method of any one of claims 23-25, wherein said candidate compound is a putative pesticidal or pharmaceutical agent.

27. The method of any one of claims 23-25, wherein said contacting comprises administering said candidate compound to cultured host cells that have been genetically engineered to express said protein.

28. The method of any one of claims 23-25, wherein said contacting comprises administering said candidate compound to a metazoan invertebrate organism that has been genetically engineered to express said protein.

29. A first animal that is an insect or a worm that has been genetically modified to express or mis-express a protein, or the progeny of said animal that has inherited said protein expression or mis-expression, wherein said protein comprises an amino acid sequence that shares at least about 80% identity with a sequence as set forth in any of SEQ ID NOS:2, 4, or 6.

30. A method for studying activity of a protein, comprising detecting the phenotype caused by the expression or mis-expression of said protein in the first animal of claim 29.

31. The method of claim 30 additionally comprising observing a second animal having the same genetic modification as said first animal which causes said expression or mis-expression of said protein, and wherein said second animal additionally comprises a mutation in a gene of interest, wherein differences, if any, between the phenotype of the first animal and the phenotype of the second animal identifies the gene of interest as capable of modifying the function of the gene encoding said protein.

32. The method of claim 30 additionally comprising administering one or more candidate compounds to said animal or its progeny and observing any changes in a biological activity associated with said protein in said animal or its progeny.
